(19) United States
(12) Patent Application Publication
(10) Pub. No.: US 2005/0265987 A1
(43) Pub. Date: Dec. 1, 2005

(54) Sulfatases and methods of use thereof

(76) Inventors: Steven Rosen, San Francisco, CA (US); Stefan Hemmerich, Berkeley, CA (US); Megumi Tomita, San Francisco, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/933,025

(22) Filed: Sep. 1, 2004

Related U.S. Application Data
(63) Continuation of application No. 10/025,966, filed on Dec. 21, 2001, now abandoned.
(60) Provisional application No. 60/267,831, filed on Feb. 9, 2001.
(60) Provisional application No. 60/258,577, filed on Dec. 27, 2000.

(51) Int. Cl. A61K 38/47; C12N 9/16; C07H 21/04; C12P 21/06

(52) U.S. Cl. 424/94.61; 435/196; 435/69.1; 435/320.1; 435/325; 536/23.2

(57) ABSTRACT

Novel sulfatases and polypeptides related thereto, as well as nucleic acid compositions encoding the same, are provided. The subject polypeptides and nucleic acid compositions find use in a variety of applications, including various diagnostic and therapeutic agent screening applications. Also provided are methods of inhibiting tumor-induced angiogenesis and methods of treating disease conditions associated therewith, particularly by administering an inhibitor of a subject sulfatase.

### CROSS-REFERENCE

This application claims the benefit of U.S. Provisional Patent Application No. 60/258,577, filed Dec. 27, 2000, and U.S. Provisional Patent Application No. 60/267,831, filed Feb. 9, 2001, both of which are incorporated herein by reference in their entirety.

### STATEMENT REGARDING FEDERALLY SPONSORED RESEARCH

This invention was made with Government support under Grant No. GM23547, awarded by the National Institutes of Health. The United States Government has certain rights in this invention.

### FIELD OF THE INVENTION

The present invention is in the field of sulfatase enzymes.

### BACKGROUND OF THE INVENTION

Sulfatase enzymes are involved in a variety of physiological processes, including development, metabolism, and inflammation. For example, the developmental signaling functions of cell surface heparan sulfate proteoglycans (HSPGs) are dependent on their sulfation states. Human lysosomal arylsulfatase A is a prototype member of the sulfatase family. Glucosamine-6-sulphatase is an exo-hydrolase required for the lysosomal degradation of heparan sulphate and keratan sulphate. These enzymes require the posttranslational oxidation of the —CH₂SH group of a conserved cysteine to an aldehyde, yielding a formylglycine. Without this modification sulfatases are catalytically inactive, as revealed by a lysosomal storage disorder known as multiple sulfatase deficiency. For example, deficiency of glucosamine-6-sulphatase activity leads to the lysosomal storage of the glycosaminoglycan, heparan sulphate and the monosaccharide sulphate N-acetylglucosamine 6-sulphate and the autosomal recessive genetic disorder mucopolysaccharidosis type IIID.

Others have isolated and identified a glycosulfatase that removes the sulfate moiety from mucous glycoprotein. Further, others have isolated and specifically identified human glucosamine-6-sulfatase and obtained cDNA coding for such. Finally, others isolated and specifically identified N-acetylgalactosamine-6-sulfate/galactose-6-sulfate sulfatase.

Angiogenesis and vasculogenesis are processes involved in the growth of blood vessels. Angiogenesis is the process by which new blood vessels are formed from extant capillaries, while vasculogenesis involves the growth of vessels deriving from endothelial progenitor cells. Angiogenesis and vasculogenesis, and the factors that regulate these processes, are important in embryonic development, inflammation, and wound healing. However, angiogenesis and vasculogenesis also contribute to pathologic conditions such as tumor growth, diabetic retinopathy, rheumatoid arthritis, and chronic inflammatory diseases (see, e.g., U.S. Pat. No. 5,318,957; Yancopoulos et al. (1998) Cell 93:661-4; Folkman et al. (1996) Cell 87; 1153-5; and Hanahan et al. (1996) Cell 86:353-64). For example, generation of new blood vessels in the vicinity of a tumor allows the tumor to grow and, in come cases, metastasize.

Several angiogenic and/or vasculogenic agents with different properties and mechanisms of action are well known in the art. For example, acidic and basic fibroblast growth factor (FGF), transforming growth factor alpha (TGF-α) and beta (TGF-β), tumor necrosis factor (TNF), platelet-derived growth factor (PDGF), vascular endothelial cell growth factor (VEGF), and angiogenin are potent and well-characterized angiogenesis-promoting agents.

Despite the availability of therapies to treat cancer, ischemic conditions, and inflammation, a need exists for additional ways to combat these disorders. The present invention addresses this need.

Literature

Parenti et al. (1997) Curr. Opinion Genet. Devel. 7:386-391; Bergers et al. (2000) Nature Cell Biol. 2:737-744; Lukatela et al. (1998) Biochem. 37:3654; Knaust et al. (1998) Biochem. 37:13941; Robertson et al. (1992) Biochem J. 288:539; Robertson et al. (1993) Biochem J. 293:683-689; Robertson et al. (1988) Biochem. Biophys. Res. Commun., 157:218-224; Tomatsu et al. (1991) Biochem. Biophys. Res. Commun. 181:677-683; Folkman et al. (1992) Seminars in Cancer Biology 3:89-96; Dhoot et al. (2001) Science 293:1663-1666. U.S. Pat. Nos. 5,925,349; and 5,695,752. International Patent Applications WO 98/53071; WO 99/54448; WO 99/63088; WO 00/06086; WO 01/00828; WO 01/02568; WO 01/40269; WO 01/42467; WO 01/59127; WO 01/57058; WO 01/21640.

### SUMMARY OF THE INVENTION

Novel sulfatases and polypeptides related thereto, as well as nucleic acid compositions encoding the same, are provided. The subject polypeptide and nucleic acid compositions find use in a variety of applications, including diagnostic applications, and therapeutic agent screening applications, as well as in treatment of a variety of disease conditions. Also provided are methods of modulating sulfatase enzymatic activity and methods of treating disease conditions associated therewith, particularly by administering inhibitors of the novel sulfatases.

### BRIEF DESCRIPTION OF THE DRAWINGS

FIGS. 1A (1Ai and 1Aii) and 1B provide the cDNA sequence and amino acid sequence, respectively, of human SULF1. The full length cDNA sequence is SEQ ID NO:01, the open reading frame is set forth in SEQ ID NO:02, and the amino acid sequence of the protein encoded by the open reading frame is SEQ ID NO:03.

FIGS. 2A (2Ai and 2Aii) and 2B provide the cDNA sequence and amino acid sequence, respectively, of human SULF2. The full length cDNA sequence is SEQ ID NO:04, the open reading frame is set forth in SEQ ID NO:05, and the amino acid sequence of the protein encoded by the open reading frame is SEQ ID NO:06.

FIGS. 3A (3Ai and 3Aii) and 3B provide the cDNA sequence and amino acid sequence, respectively, of mouse SULF-1. The full length cDNA sequence is SEQ ID NO:07, the open reading frame is set forth in SEQ ID NO:08, and the amino acid sequence of the protein encoded by the open reading frame is SEQ ID NO:09.

FIGS. 4A (4Ai and 4Aii) and 4B provide the cDNA sequence and amino acid sequence, respectively, of mouse SULF-2. The full length cDNA sequence is SEQ ID NO:10, the open reading frame is set forth in SEQ ID NO:11, and the amino acid sequence of the protein encoded by the open reading frame is SEQ ID NO:12.

FIG. 5 is a graph depicting the numbers of human SULF1 expressed sequence tags (ESTs) in normal and tumor tissue libraries.

FIG. 6 is a graph depicting the numbers of huSULF1 ESTs in various tissues.

FIG. 7 is a graph depicting the numbers of human SULF2 expressed sequence tags (ESTs) in normal and tumor tissue libraries.

FIG. 8 depicts the results of SAGE analysis of huSULF-1 in normal and cancer tissues.

FIG. 9 depicts the results of SAGE analysis of huSULF-2 in normal and cancer tissues.

FIGS. 10A (10Ai and 10Aii) and 10B provide the cDNA sequence and amino acid sequence, respectively of human SULF-2. The full length cDNA sequence is SEQ ID NO:13, the open reading frame is set forth in SEQ ID NO:14, and the amino acid sequence of the protein encoded by the open reading frame is SEQ ID NO:15.

FIGS. 11A (11Ai and 11Aii) and 11B provide the cDNA sequence and amino acid sequence, respectively of mouse SULF-2. The full length cDNA sequence is SEQ ID NO:16, the open reading frame is set forth in SEQ ID NO:17, and the amino acid sequence of the protein encoded by the open reading frame is SEQ ID NO:18.

FIG. 12 depicts exon start and end sites, and exon length for human SULF2 gene exons.

FIG. 13 is a schematic representation of human sulf-1 and sulf-2 protein domain.

FIG. 14 is a schematic representation of an activity of a subject sulfatase.

### DEFINITIONS

The terms “polynucleotide” and “nucleic acid molecule” are used interchangeably herein to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes single-, double-stranded and triple helical molecules. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as oligomers or oligos and may be isolated from genes, or chemically synthesized by methods known in the art.

The following are non-limiting embodiments of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules, such as methylated nucleic acid molecules and nucleic acid molecule analogs. Analogs of purines and pyrimidines are known in the art. Nucleic acids may be naturally occurring, e.g. DNA or RNA, or may be synthetic analogs, as known in the art. Such analogs may be preferred for use as probes because of superior stability under assay conditions. Modifications in the native structure, including alterations in the backbone, sugars or heterocyclic bases, have been shown to increase intracellular stability and binding affinity. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage.

Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity.

Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

The terms “polypeptide” and “protein”, used interchangebly herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

A “substantially isolated” or “isolated” polynucleotide is one that is substantially free of the sequences with which it is associated in nature. By substantially free is meant at least 50%, preferably at least 70%, more preferably at least 80%, and even more preferably at least 90% free of the materials with which it is associated in nature. As used herein, an “isolated” polynucleotide also refers to recombinant polynucleotides, which, by virtue of origin or manipulation: (1) are not associated with all or a portion of a polynucleotide with which it is associated in nature, (2) are linked to a polynucleotide other than that to which it is linked in nature, or (3) does not occur in nature.

Hybridization reactions can be performed under conditions of different “stringency”. Conditions that increase stringency of a hybridization reaction of widely known and published in the art. See, for example, Sambrook et al. (1989). Examples of relevant conditions include (in order of increasing stringency): incubation temperatures of 25° C., 37° C., 50° C. and 68° C.; buffer concentrations of 10×SSC, 6×SSC, 1×SSC, 0.1×SSC (where 1×SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or deionized water. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 1×SSC (150 mM NaCl, 15 mM sodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C. Stringent hybridization conditions are hybridization conditions that are at least as stringent as the above representative conditions. Other stringent hybridization conditions are known in the art and may also be employed to identify nucleic acids of this particular embodiment of the invention.

“Tₘ” is the temperature in degrees Celsius at which 50% of a polynucleotide duplex made of complementary strands hydrogen bonded in anti-parallel direction by Watson-Crick base pairing dissociates into single strands under conditions of the experiment. Tₘ may be predicted according to a standard formula, such as:
Tₘ=81.5+16.6 log [X⁺]+0.41 (% G/C)−0.61(% F)−600/L
where [X⁺] is the cation concentration (usually sodium ion, Na⁺) in mol/L; (% G/C) is the number of G and C residues as a percentage of total residues in the duplex; (% F) is the percent formamide in solution (wt/vol); and L is the number of nucleotides in each strand of the duplex.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at http://ww.ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970)

Of interest is the BestFit program using the local homology algorithm of Smith Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters:
Mismatch Penalty: 1.00;
Gap Penalty: 1.00;
Gap Size Penalty: 0.33; and
Joining Penalty: 30.0.

One parameter for determining percent sequence identity is the “percentage of the alignment region length” where the strongest alignment is found.

The percentage of the alignment region length is calculated by counting the number of residues of the individual sequence found in the region of strongest alignment. This number is divided by the total residue length of the target or query polynucleotide sequence to find a percentage. An example is shown below:

| Target sequence: | GCGCGAAATACTCACTCGAGG |
|---|---|
| | \| \|\|\| \|\|\|\| \|\|\| |
| Query sequence: | TATAGCCCTAC.CACTAGAGTCC |
| | 1 5 10 15 |

The region of alignment begins at residue 9 and ends at residue 19. The total length of the target sequence is 20 residues. The percent of the alignment region length is 11 divided by 20 or 55%, for example.

Percent sequence identity is calculated by counting the number of residue matches between the target and query polynucleotide sequence and dividing total number of matches by the number of residues of the target or query sequence found in the region of strongest alignment. For the example above, the percent identity would be 10 matches divided by 11 residues, or approximately, 90.9%

The percent of the alignment region length is typically at least about 55% of total length of the sequence, more typically at least about 58%, and even more typically at least about 60% of the total residue length of the sequence. Usually, percent length of the alignment region can be as great as about 62%, more usually as great as about 64% and even more usually as great as about 66%.

Stringent conditions for both DNA/DNA and DNA/RNA hybridization are as described by Sambrook et al. Molecular Cloning, A Laboratory Manual, 2nd Ed., Cold Spring Harbor Laboratory Press, Cold Spring Harbor, N.Y., 1989, herein incorporated by reference. For example, see page 7.52 of Sambrook et al.

The term “host cell” includes an individual cell or cell culture which can be or has been a recipient of any recombinant vector(s) or isolated polynucleotide of the invention. Host cells include progeny of a single host cell, and the progeny may not necessarily be completely identical (in morphology or in total DNA complement) to the original parent cell due to natural, accidental, or deliberate mutation and/or change. A host cell includes cells tranfected or infected in vivo or in vitro with a recombinant vector or a polynucleotide of the invention. A host cell which comprises a recombinant vector of the invention is a “recombinant host cell.”

The term “binds specifically,” in the context of antibody binding, refers to high avidity and/or high affinity binding of an antibody to a specific polypeptide i.e., epitope of a sulfatase polypeptide. Antibody binding to an epitope on a specific sulfatase polypeptide (also referred to herein as “a sulfatase epitope”) is preferably stronger than binding of the same antibody to any other epitope, particularly those which may be present in molecules in association with, or in the same sample, as the specific polypeptide of interest, e.g., binds more strongly to a specific sulfatase epitope than to a different sulfatase epitope so that by adjusting binding conditions the antibody binds almost exclusively to the specific sulfatase epitope and not to any other sulfatase epitope, and not to any other sulfatase polypeptide which does not comprise the epitope. Antibodies which bind specifically to a subject polypeptide may be capable of binding other polypeptides at a weak, yet detectable, level (e.g., 10% or less of the binding shown to the polypeptide of interest). Such weak binding, or background binding, is readily discernible from the specific antibody binding to a subject polypeptide, e.g. by use of appropriate controls. In general, antibodies of the invention which bind to a specific sulfatase polypeptide with a binding affinity of 10⁻⁷ M or more, preferably 10⁻⁸ M or more (e.g., 10⁻⁹ M, 10⁻¹⁰, 10⁻¹¹, etc.). In general, an antibody with a binding affinity of 10⁻⁶ M or less is not useful in that it will not bind an antigen at a detectable level using conventional methodology currently used.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “biological sample” encompasses a clinical sample, and also includes cells in culture, cell supernatants, cell lysates, serum, plasma, biological fluid, and tissue samples.

The term “angiogenesis” refers to a process of tissue vascularization that involves the development of new vessels. Angiogenesis occurs via one of three mechanisms: (1) neovascularization, where endothelial cells migrate out of pre-existing vessels beginning the formation of the new vessels; (2) vasculogenesis, where the vessels arise from precursor cells de novo; or (3) vascular expansion, where existing small vessels enlarge in diameter to form larger vessels (Blood, et al. (1990) Biochem. Biophys. Acta. 1032:89-118).

The terms “cancer”, “neoplasm”, “tumor”, and “carcinoma”, are used interchangeably herein to refer to cells which exhibit relatively autonomous growth, so that they exhibit an aberrant growth phenotype characterized by a significant loss of control of cell proliferation. Cancerous cells can be benign or malignant.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines, simians, humans, felines, canines, equines, bovines, mammalian farm animals, mammalian sport animals, and mammalian pets.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a sulfatase” includes a plurality of such sulfatases and reference to “the agent” includes reference to one or more agents and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

Overview

Sulfatases are a family of enzymes that release sulfate from glycoproteins, sulfolipids, and proteoglycans. The present invention provides novel sulfatases and polypeptides related thereto, as well as nucleic acid compositions encoding the same. The subject polypeptide and/or nucleic acid compositions find use in a variety of different applications, including various diagnostic and therapeutic agent screening/discovery/preparation applications.

In many embodiments, a novel sulfatase of the invention exhibits one or more of the following properties: (1) exhibits glucosamine-6-sulfatase activity; (2) is an endosulfatase, removing sulfate from the C-6 position of internal glucosamines as well as from glucosamines at the non-reducing termini of polysaccharides (3) removes a sulfate group from glycoproteins and/or proteoglycans; (4) is secreted from a eukaryotic cell; (5) acts on extracellular matrix (ECM) components to remove a sulfate group, resulting in release from the ECM of extracellular differentiation factors and/or growth factors; (6) mRNA encoding the sulfatase shows elevated expression in tumors; and (7) is secreted in greater abundance from a cancerous cell as compared to a non-cancerous cell of the same cell and/or tissue type.

The subject sulfatases are expressed at elevated levels in tumors, compared with normal tissue. Without wishing to be bound by any particular theory, it is believed that a subject sulfatase is secreted from a tumor cell, and acts on component(s) of the ECM to release one or more differentiation factors or growth factors, including angiogenic factor(s). Angiogenic factors then act on local endothelial cells and promote angiogenesis, resulting in access of the tumor to the vasculature, and therefore to the blood supply. By reducing access of a tumor to the vasculature, one can reduce tumor growth.

Polypeptide Compositions

Novel sulfatases, as well as polypeptide compositions related thereto, are provided. The invention provides a sulfatase present in other than its natural environment. Novel sulfatases of the invention encompass SULF1 and SULF2. In some embodiments, a subject sulfatase is a human sulfatase. In other embodiments, a subject sulfatase is a mouse sulfatase.

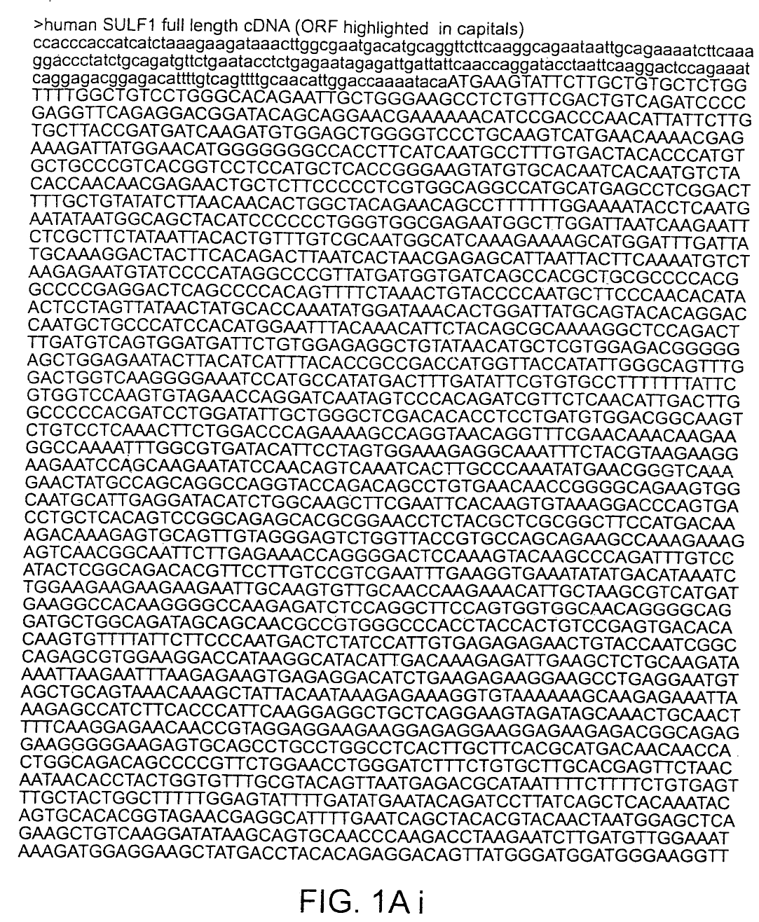

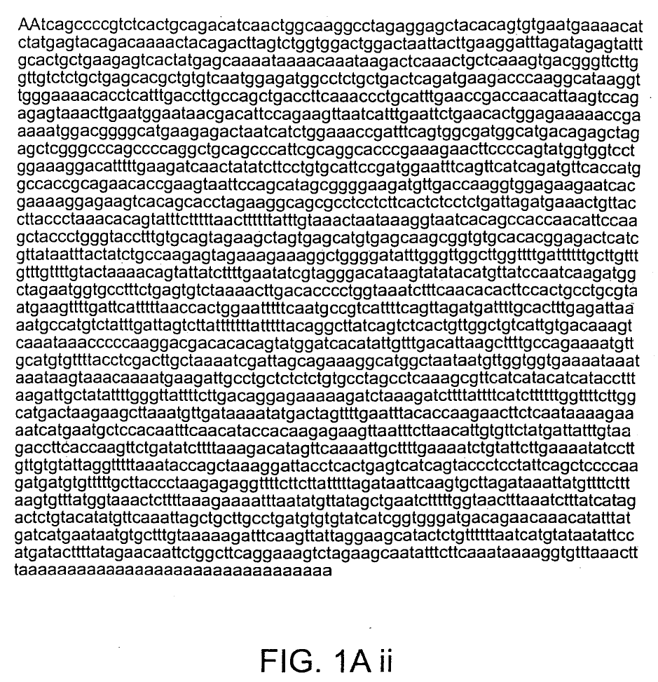

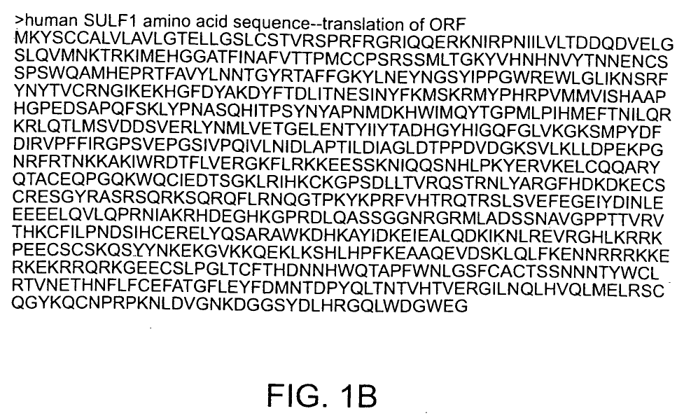

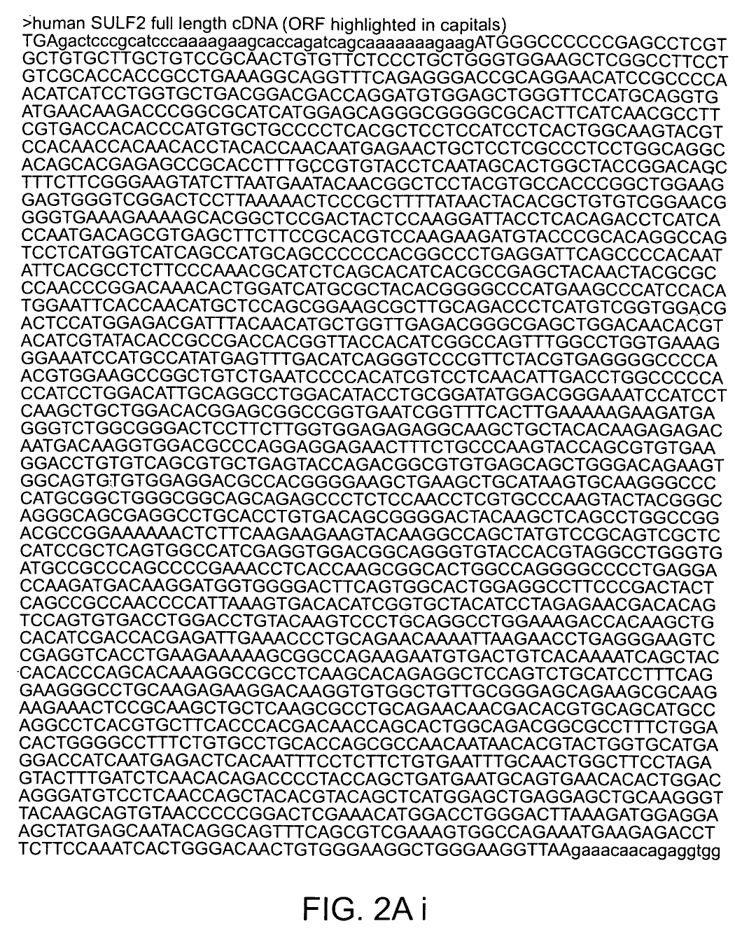

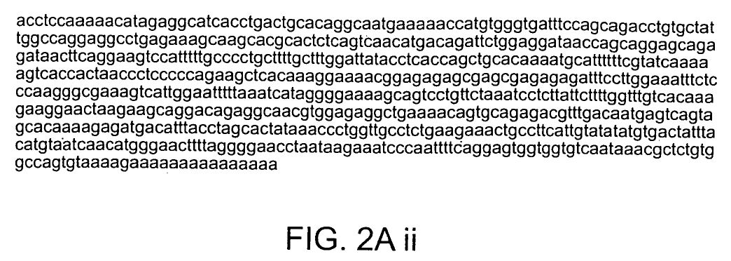

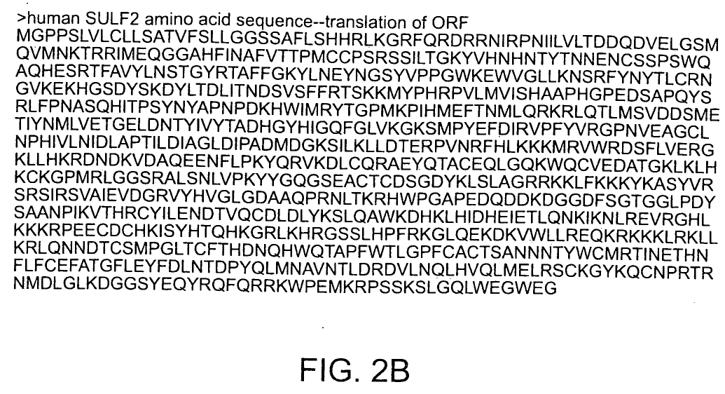

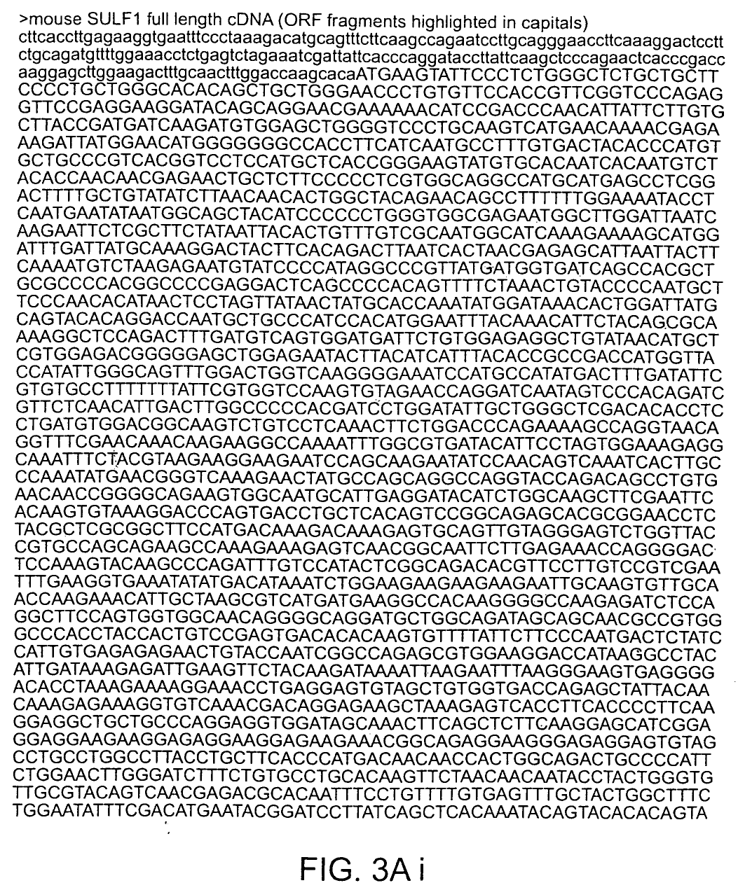

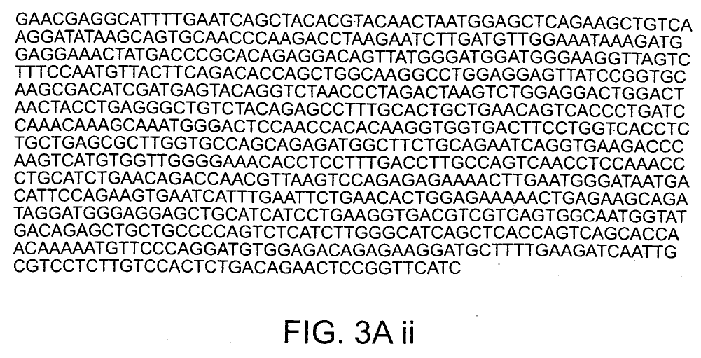

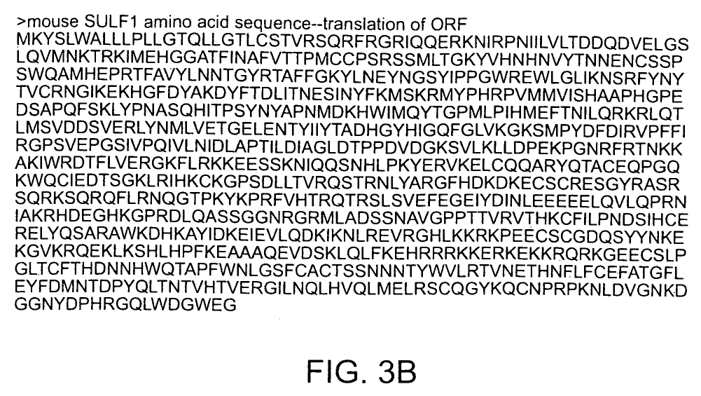

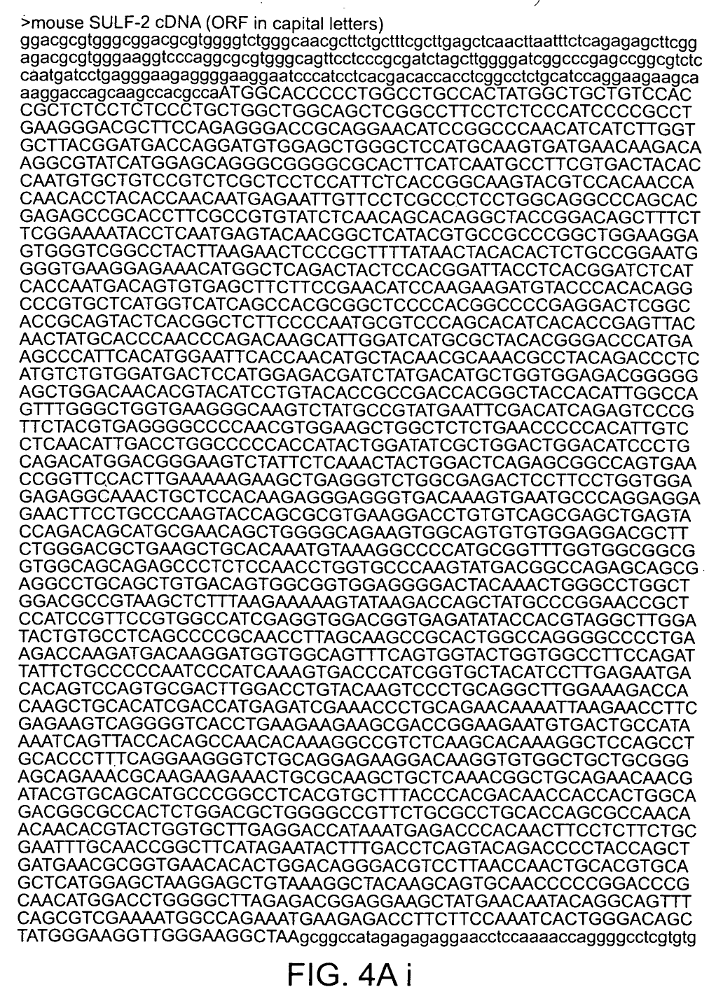

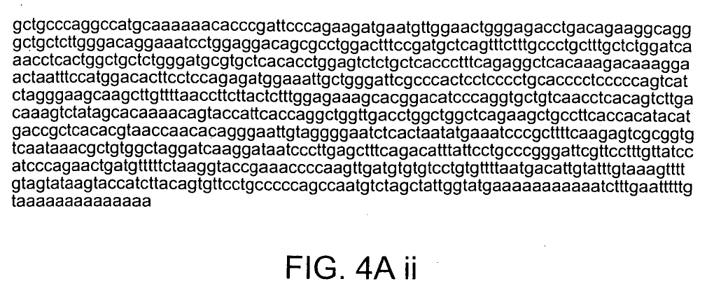

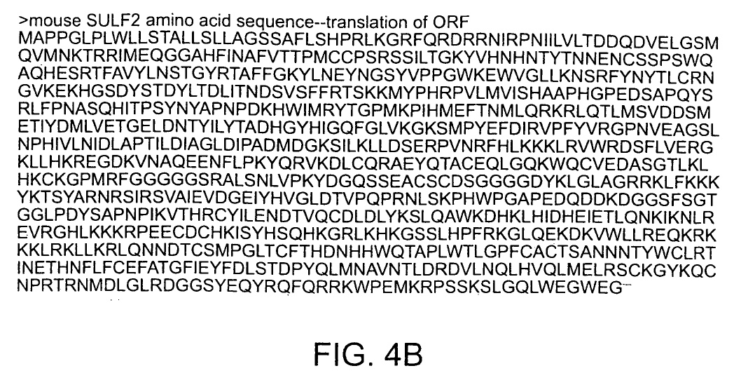

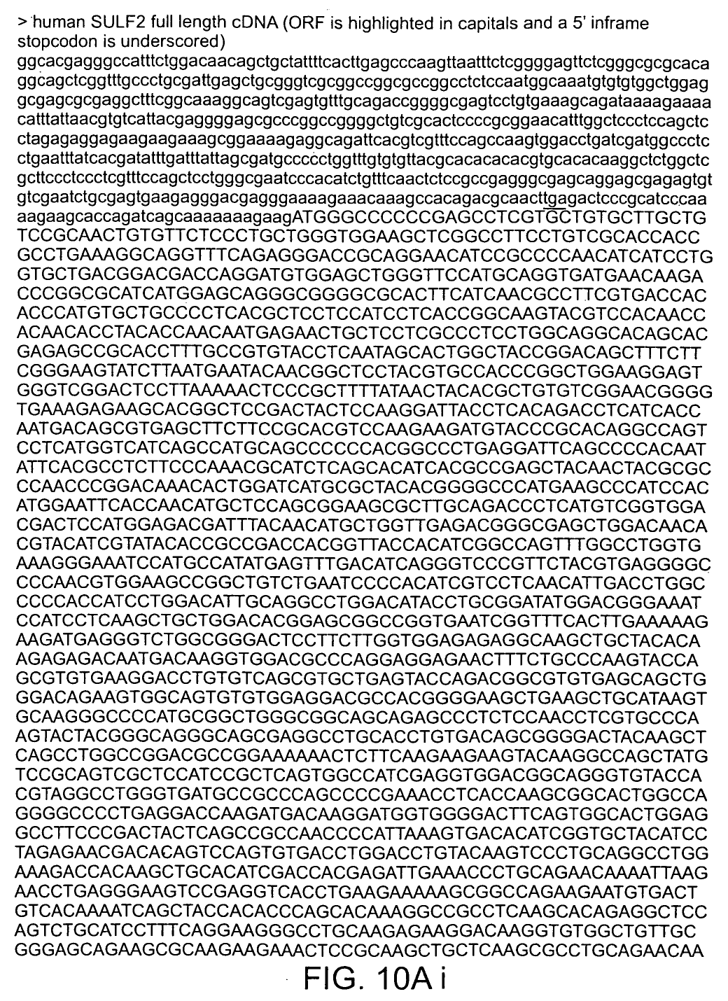

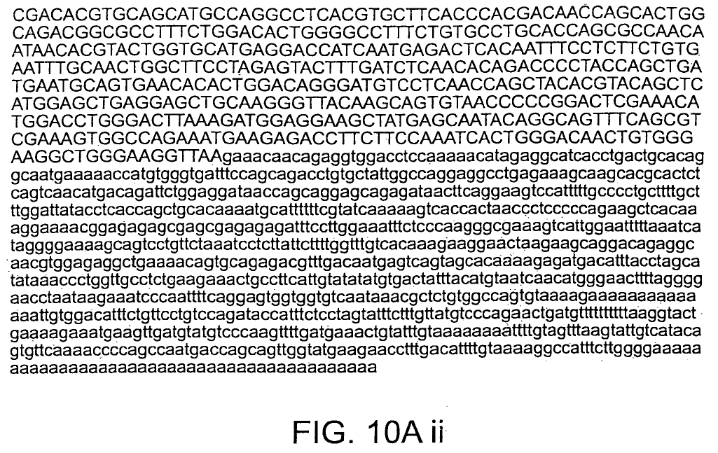

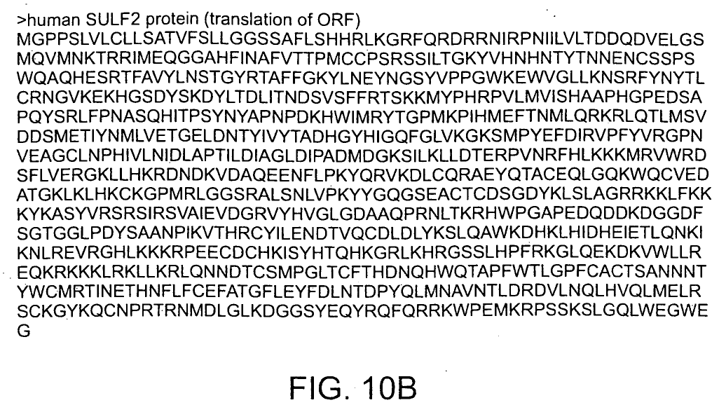

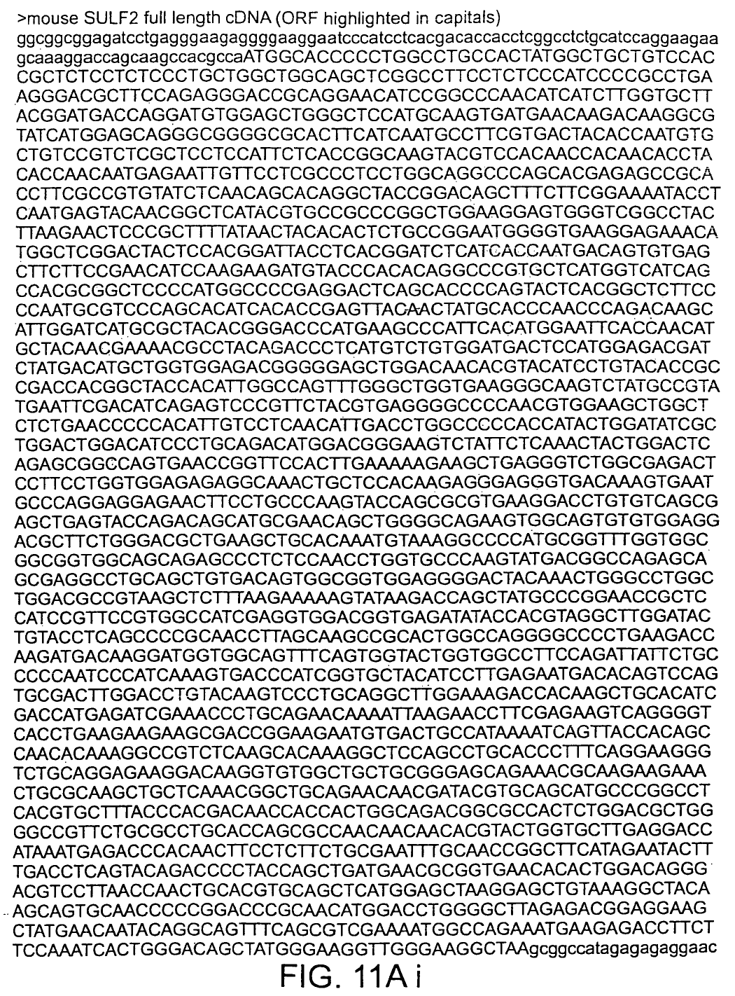

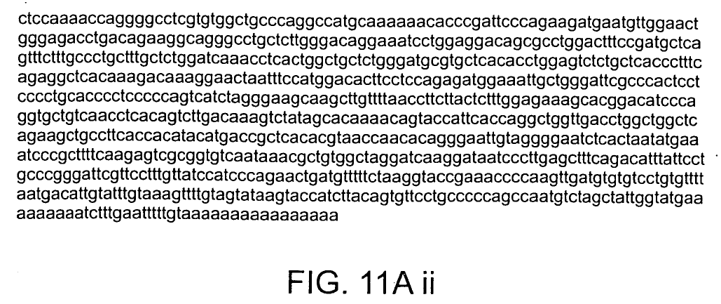

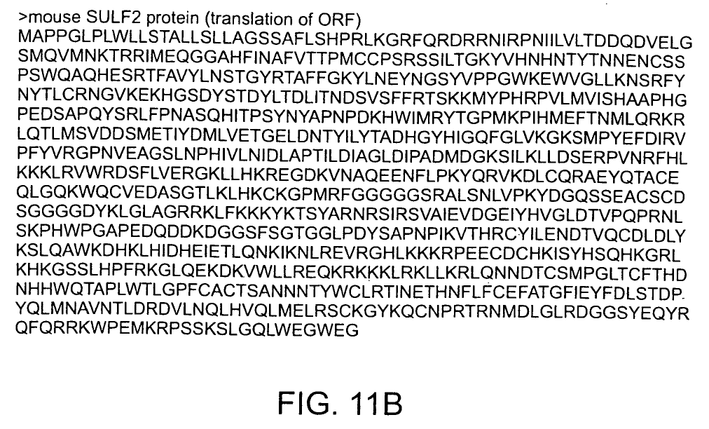

In particular embodiments, a subject sulfatase has an amino acid sequence substantially identical to the sequence of any one of SEQ ID NOS:03, 06, 09, 12, 15, and
18. In other particular embodiments, a subject sulfatase has an amino acid sequence substantially identical to any one of the sequences depicted in FIG. 1B, FIG. 2B, FIG. 3B, FIG. 4B, FIG. 10B, and FIG. 11B.

In many embodiments, a novel sulfatase of the invention exhibits one or more of the following properties: (1) exhibits glucosamine-6-sulfatase activity; (2) is an endosulfatase, removing sulfate from the C-6 position of internal glucosamines as well as from glucosamines at the non-reducing termini of polysaccharides (3) removes a sulfate group from glycoproteins and/or proteoglycans; (4) is secreted from a eukaryotic cell; (5) acts on extracellular matrix (ECM) components to remove a sulfate group, resulting in release from the ECM of extracellular differentiation factors and/or growth factors; (6) mRNA encoding the sulfatase shows elevated expression in tumors; and (7) is secreted in greater abundance from a cancerous cell as compared to a non-cancerous cell of the same cell and/or tissue type.

The invention also provides fragments of the subject sulfatases. In some embodiments, fragments exhibit sulfatase activity. Fragments find utility in generating antibodies to the full-length sulfatases; and in methods of screening for candidate agents that bind to and/or modulate sulfatase enzymatic activity. The term “sulfatase polypeptide composition” as used herein refers to both the full-length human protein as well as portions or fragments thereof. Also included in this term are variations of the naturally occurring human protein, where such variations are homologous or substantially similar to the naturally occurring protein, as described in greater detail below, as well as corresponding homologs from non-human species, such as other mammalian species. In the following description of the subject invention, the terms “SULF1” and “SULF2” are used to refer not only to the human form of these novel sulfatases, but also to homologs thereof expressed in non-human species.

Human SULF1 (huSULF1) is an 871 amino acid protein having an amino acid sequence as shown in FIG. 1B and identified as SEQ ID NO:03. HuSULF1 has a molecular weight based on its amino acid of about 80 to about 100 kDa.

Human SULF2 (huSULF2) is an 870 amino acid protein having an amino acid sequence as shown in FIG. 2B and identified as SEQ ID NO:06. HuSULF2 has a molecular weight based on its amino acid of about 80 to about 100 kDa. In some embodiments, a subject sulfatase has an amino acid sequence as shown in FIG. 10B and as set forth in SEQ ID NO:15.

Mouse SULF1 (mSULF1) is an 870 amino acid protein having an amino acid sequence as shown in FIG. 3B and as set forth in SEQ ID NO:09.

Mouse SULF2 (mSULF2) is an 875 amino acid protein having an amino acid sequence as shown in FIG. 4B and as set forth in SEQ ID NO:12. In some embodiments, a subject sulfatase has an amino acid sequence as shown in FIG. 11B and as set forth in SEQ ID NO:18.

The subject sulfatases have a molecular weight of between 80 and 100 kDa based on their amino acid sequences. Subject sulfatases produced by a eukaryotic cell are glycosylated, and in some embodiments have a molecular weight of about 126 kDa. In addition, in some embodiments, a subject sulfatase is proteolytically cleaved to produce fragments of from about 60 kDa to about 70 kDa (e.g., 61 kDa, 66 kDa, 71 kDa); from about 48 kDa to about 55 kDa (e.g., 49 kDa, 53 kDa); or from about 40 to about 55 kDa (e.g., 40 kDa, 49 kDa, 53 kDa). Many of these fragments or associations of these fragments have sulfatase activity.

In addition to the above specifically listed proteins, sulfatases from other species are also provided, including mammals, such as: rodents, e.g. mice, rats; domestic animals, e.g. horse, cow, dog, cat; and humans, as well as non-mammalian species, e.g. avian, and the like. By homolog is meant a protein having at least about 35%, at least about 40%, at least about 60%, at least about 70%, at least about 75%, at least about 80%, at least about 90%, or at least about 95%, or higher, amino acid sequence identity to the one of the above specifically listed sulfatases, as measured by using the “GAP” program (part of the Wisconsin Sequence Analysis Package available through the Genetics Computer Group, Inc. (Madison Wis.)), where the parameters are: Gap weight:12; length weight:4. In many embodiments of interest, homology will be at least 75, usually at least 80 and more usually at least 85%, where in certain embodiments of interest homology will be as high as 90%.

Also provided are sulfatase proteins that are substantially identical to the above listed proteins, where by substantially identical is meant that the protein has an amino acid sequence identity to the sequence one of the above listed proteins of at least about 75%, at least about 80% at least about 85%, at least about 90%, at least about 95%, or at least about 98%.

The proteins of the subject invention (e.g. SULF1, SULF2, huSULF1, huSULF2, mSULF1, mSULF2, and the like) are present in a non-naturally occurring environment, e.g. are separated from their naturally occurring environment. In certain embodiments, the subject proteins are present in a composition that is enriched for subject protein as compared to its naturally occurring environment. For example, purified sulfatases are provided, where by purified is meant that the sulfatase is present in a composition that is substantially free of non-sulfatase proteins, where by substantially free is meant that less than 90%, usually less than 60% and more usually less than 50% of the composition is made up of non-sulfatase proteins.

The proteins of the subject invention may also be present as an isolate, by which is meant that the protein is substantially free of other proteins and other naturally occurring biologic molecules, such as oligosaccharides, polynucleotides and fragments thereof, and the like, where substantially free in this instance means that less than 70%, usually less than 60% and more usually less than 50% of the composition containing the isolated protein is some other naturally occurring biological molecule. In certain embodiments, the proteins are present in substantially pure form, where by substantially pure form is meant at least 95%, usually at least 97% and more usually at least 99% pure.

In addition to the naturally occurring proteins, polypeptides which vary from the naturally occurring proteins (e.g., huSULF1, huSULF2, mSULF1, mSULF2, etc.) are also provided. By SULF1 and SULF2 polypeptide is meant an amino acid sequence encoded by an open reading frame (ORF) of the SULF1 and SULF2 gene, described in greater detail below, including the full length SULF1 and SULF2 protein and fragments thereof, particularly biologically active fragments and/or fragments corresponding to functional domains, e.g., sulfatase active site; and including fusions of the subject polypeptides to other proteins or parts thereof. Fusion proteins may comprise a subject polypeptide, or fragment thereof, and a non-SULF polypeptide (“the fusion partner”) fused in-frame at the N-terminus and/or C-terminus of the subject SULF polypeptide.

Fusion partners include, but are not limited to, polypeptides that can bind antibody specific to the fusion partner (e.g., epitope tags, e.g., hemagglutinin (HA; e.g., CYPYDVPDYA; SEQ ID NO:19), FLAG (e.g., DYKDDDDK; SEQ ID NO:20), c-myc (e.g., CEQKLISEEDL; SEQ ID NO:21), and the like); polypeptides that provide a detectable signal (e.g., a fluorescent protein, e.g., a green fluorescent protein, a fluorescent protein from an Anthozoan species; β-galactosidase; luciferase; and the like);
polypeptides that provide a catalytic function or induce a cellular response; polypeptides that provide for secretion of the fusion protein from a eukaryotic cell; polypeptides that provide for secretion of the fusion protein from a prokaryotic cell; polypeptides that provide for binding to metal ions (e.g., Hisₙ, where n=3-10, e.g., 6His); and the like.

In some embodiments, a SULF polypeptide of the invention comprises at least about 10, at least about 20, at least about 25, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, or at least about 850 contiguous amino acids of one of the sequences as set forth in any one of SEQ ID NOS:3, 6, 9, 12, 15, and 18, and in FIGS. 1B, 2B, 3B, 4B, 10B, and 11B, up to the entire amino acid sequence as set forth in any one of SEQ ID NOS:3, 6, 9, 12, 15, and 18, and in FIGS. 1B, 2B, 3B, 4B, 10B, and 11B.

Fragments of the subject polypeptides, as well as polypeptides comprising such fragments, are also provided. Fragments of SULF1 and SULF2 of interest will typically be at least about 10 amino acids (aa) in length, usually at least about 50 aa in length, and may be as long as 300 aa in length or longer, where the fragment will have a stretch of amino acids that is identical to the subject protein of at least about 10 aa, and usually at least about 15 aa, and in many embodiments at least about 50 aa in length.

Specific fragments of interest include the first sulfatase domain; a hydrophilic domain; and a second sulfatase domain. The first sulfatase domain encompasses from about amino acid 42 to about amino acid 389; the hydrophilic domains are about 370 amino acids in length and encompass from about amino acid 370 to about 740; and the second sulfatase domain is approximately 70 amino acids in length and encompasses from about amino acid 766 to about amino acid 837. The first sulfatase domain cleaves the sulfate moiety from N-acetylglucosamine-6-sulfate or glucosamine-6-sulfate structures within heparan sulfate glycosamino glycans and related glycoconjugates. The hydrophilic domain binds to the cell surface or to substrates for the enzyme. The second sulfatase domain is involved in sulfate recognition of glucosamine and N-acetyl glucosamine sugars.

Accordingly, in some embodiments, a subject sulfatase fragment is from about amino acid 40 to about amino acid 390, from about amino acid 370 to about amino acid 740, or from about amino acid 760 to about amino acid 840 of any one of SEQ ID NOs:03, 06, 09, 12, 15, or 18, or variants thereof, especially variants containing conserved amino acid substitutions. The invention provides polypeptides comprising such fragments, including, e.g., fusion polypeptides comprising a subject sulfatase fragment fused in frame (directly or indirectly) to a heterologous protein. Suitable heterologous proteins include, but are not limited to, a protein that serves as a detectable marker (e.g., a fluorescent protein, β-galactosidase, luciferase); an immunologically detectable protein (e.g., an epitope tag); and a structural protein.

Within the first sulfatase domains are cleavage sites for the furan/PACE protease processing enzymes. This cleavage occurs between residues 408 (arginine) and 409 (aspartic acid) and/or between 576 (arginine) and 577 (histidine) of hsulf-1. The cleavage occurs between 409 (arginine) and 410 (aspartic acid) and/or between 423 (arginine) and 424 (aspartic acid) and/or between 538 (arginine) and 539 (serine) and/or between 565 (arginine) and 566 (histidine) of hsulf-2. Cleavage is necessary for activity of the enzyme. Accordingly, in many embodiments, a subject sulfatase is cleaved at one or more furan/PACE cleavage sites. Thus, in many embodiments, a subject sulfatase includes amino acids from about 1 to about amino acid 408 or 409 (e.g., up to the first furan/PACE cleavage site).

Sulfatase fragments, such as those described above, are useful in screening assays, to identify agents that modulate an activity of a subject sulfatase. Screening assays are described in more detail below. For example, a polypeptide comprising a first sulfatase domain is used in a screening assay to identify agents that modulate cleavage of the sulfate moiety from the N-acetylglusamine-6-sulfate or glucosamine-6-sulfate structures within heparan sulfate glycosamino glycans and related glycoconjugates. A polypeptide comprising the hydrophilic domain is used in a screening assay to identify agents that modulate binding of the domain to negatively charged surface structures on the surface of cells, such as proteoglycans. A polypeptide comprising the second sulfatase domain is used in a screening assay to identify agents that modulate sulfate recognition of glucosamine and N-acetyl glucosamine sugars. Polypeptides that comprise sulfatase fragments include polypeptides that include a fusion partner fused in-frame at the amino and/or carboxyl terminus of the sulfatase fragment.

The subject proteins and polypeptides may be obtained from naturally occurring sources or synthetically produced. Where obtained from naturally occurring sources, the source chosen will generally depend on the species from which the protein is to be derived. The subject proteins may also be derived from synthetic means, e.g. by expressing a recombinant gene encoding protein of interest in a suitable host, as described in greater detail below. Any convenient protein purification procedures may be employed, where suitable protein purification methodologies are described in Guide to Protein Purification, (Deuthser ed.) (Academic Press, 1990). For example, a lysate may be prepared from the original source and purified using HPLC, exclusion chromatography, gel electrophoresis, affinity chromatography, and the like.

Nucleic Acid Compositions

Also provided are nucleic acid compositions encoding the subject novel sulfatases or fragments thereof. By nucleic acid composition is meant a composition comprising a sequence of DNA having an open reading frame that encodes one the subject sulfatases and is capable, under appropriate conditions, of being expressed as one of the subject sulfatases described above. Thus, the term encompasses genomic DNA, cDNA, mRNA, and vectors comprising the subject nucleic acid sequences. Also encompassed in this term are nucleic acids that are homologous or substantially similar or identical to the nucleic acids encoding the subject sulfatase proteins. Thus, the subject invention provides genes encoding huSULF1, huSULF2, mSULF1, mSULF2, and homologs thereof.

The human SULF1 cDNA has the nucleic acid sequence shown in FIG. 1Ai-1Aii, and identified as SEQ ID NO:01. The coding region is depicted by nucleotides shown in upper case letters in FIG. 1Ai-1Aii. The coding region is set forth in SEQ ID NO:02.

The human SULF2 cDNA has the nucleic acid sequence shown in FIG. 2Ai-2Aii, and identified as SEQ ID NO:04. The coding region is depicted by nucleotides shown in upper case letters in FIG. 2Ai-2Aii. The coding region is set forth in SEQ ID NO:05.

In some embodiments, a human SULF2 cDNA has the nucleic acid sequence shown in FIG. 11Ai-11Aii and set forth in SEQ ID NO:13, with the open reading frame (coding region) set forth in SEQ ID NO:14.

The mouse SULF1 cDNA has the nucleic acid sequence shown in FIG. 3Ai-3Aii, and identified as SEQ ID NO:07. The coding region is depicted by nucleotides shown in upper case letters in FIG. 3Ai-3Aii. The coding region is set forth in SEQ ID NO:08.

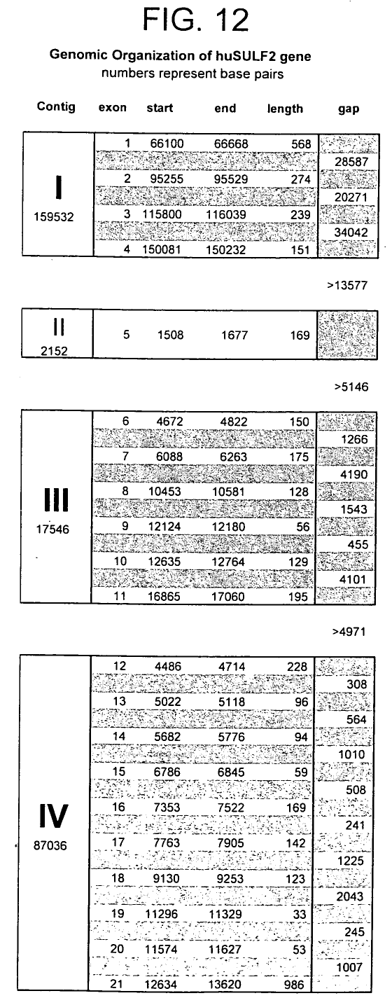

The mouse SULF2 cDNA has the nucleic acid sequence shown in FIG. 4Ai-4Aii, and identified as SEQ ID NO:10. The coding region is depicted by nucleotides shown in upper case letters in FIG. 4Ai-4Aii. The coding region is set forth in SEQ ID NO:11. In some embodiments, a mouse SULF2 cDNA has the nucleic acid sequence shown in FIG. 12Ai-12Aii, and set forth in SEQ ID NO:16, with the open reading frame set forth in SEQ ID NO:17.

In some embodiments, a SULF polynucleotide of the invention comprises a nucleotide sequence of at least about 30, at least about 50, at least about 75, at least about 100, at least about 150, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, at least about 1800, at least about 1900, at least about 2000, at least about 2100, at least about 2200, at least about 2300, at least about 2400, at least about 2500, or at least about 2600 contiguous nucleotides of the sequence set forth in one of SEQ ID NOS:1, 4, 7, 10, 13, or 16; or as set forth in any one of SEQ ID NOS:2, 5, 8, 11, 14, or 17; or in one of FIG. 1Ai-1Aii, 2Ai-2Aii, 3Ai-3Aii, 4Ai-4Aii, 11Ai-11Aii, or 12Ai-12Aii.

In some embodiments, a SULF polynucleotide of the invention specifically excludes the sequences set forth in one or more of SEQ ID NO:01, 02, 04, 05, 13, and 14.

In other embodiments, a SULF polynucleotide of the invention comprises a nucleotide sequence that encodes a polypeptide comprising an amino acid sequence of at least about 10, at least about 20, at least about 25, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, or at least about 850 contiguous amino acids of one of the sequences as set forth in any one of SEQ ID NOS:3, 6, 9, 12, 15, or 18, or as depicted in one or FIGS. 1B, 2B, 3B, 4B, 10B, and 11B, up to the entire amino acid sequence as set forth in one of SEQ ID NOS:3, 6, 9, 12, 15, or 18, or as depicted in one or FIGS. 1B, 2B, 3B, 4B, 10B, and 11B.

The source of homologous genes may be any species, e.g., primate species, particularly human; rodents, such as rats and mice, canines, felines, bovines, ovines, equines, yeast, nematodes, etc. Between mammalian species, e.g., human and mouse, homologs have substantial sequence similarity, e.g. at least 60% sequence identity, usually at least 75%, more usually at least 80% between nucleotide sequences. In many embodiments of interest, homology will be at least 75, usually at least 80 and more usually at least 85%, where in certain embodiments of interest homology will be as high as 90%. Sequence similarity is calculated based on a reference sequence, which may be a subset of a larger sequence, such as a conserved motif, coding region, flanking region, etc. A reference sequence will usually be at least about 18 nt long, more usually at least about 30 nt long, and may extend to the complete sequence that is being compared. Algorithms for sequence analysis are known in the art, such as BLAST, described in Altschul et al. (1990), J. Mol. Biol. 215:403-10 (using default settings). The sequences provided herein are essential for recognizing related and homologous proteins in database searches.

Nucleic acids encoding the proteins and polypeptides of the subject invention may be cDNA or genomic DNA or a fragment thereof. The term gene shall be intended to mean the open reading frame encoding specific proteins and polypeptides of the subject invention, and introns, as well as adjacent 5′ and 3′ non-coding nucleotide sequences involved in the regulation of expression, up to about 20 kb beyond the coding region, but possibly further in either direction. The gene may be introduced into an appropriate vector for extrachromosomal maintenance or for integration into a host genome.

The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a protein according to the subject invention.

A genomic sequence of interest comprises the nucleic acid present between the initiation codon and the stop codon, as defined in the listed sequences, including all of the introns that are normally present in a native chromosome. It may further include the 3′ and 5′ untranslated regions found in the mature mRNA. It may further include specific transcriptional and translational regulatory sequences, such as promoters, enhancers, etc., including about 1 kb, but possibly more, of flanking genomic DNA at either the 5′ or 3′ end of the transcribed region. The genomic DNA may be isolated as a fragment of 100 kbp or smaller; and substantially free of flanking chromosomal sequence. The genomic DNA flanking the coding region, either 3′ or 5′, or internal regulatory sequences as sometimes found in introns, contains sequences required for proper tissue and stage specific expression.

The genomic sequence of human SULF2 is set forth in SEQ ID NO:22. The genomic sequence of human SULF1 is set forth in SEQ ID NO:23. The genomic sequence of mouse SULF2 is set forth in SEQ ID NO:24. In particular embodiments, a subject genomic sequence has the sequence as set forth in any one of SEQ ID NO:22, 23, or 24.

The nucleic acid compositions of the subject invention may encode all or a part of the subject proteins. Double or single stranded fragments may be obtained from the DNA sequence by chemically synthesizing oligonucleotides in accordance with conventional methods, by restriction enzyme digestion, by PCR amplification, etc. For the most part, DNA fragments will be of at least 15 nt, usually at least 18 nt or 25 nt, and may be at least about 50 nt.

SULF nucleic acid molecules of the invention may comprise other, non-SULF nucleic acid molecules (“heterologous nucleic acid molecules”) of any length. For example, the subject nucleic acid molecules may be flanked on the 5′ and/or 3′ ends by heterologous nucleic acid molecules of from about 1 nt to about 10 nt, from about 10 nt to about 20 nt, from about 20 nt to about 50 nt, from about 50 nt to about 100 nt, from about 100 nt to about 250 nt, from about 250 nt to about 500 nt, or from about 500 nt to about 1000 nt, or more in length. For example, when used as a probe to detect nucleic acid molecules capable of hybridizing with the subject nucleic acids, the subject nucleic acid molecules may be flanked by heterologous sequences of any length.

The subject nucleic acid molecules may also be provided as part of a vector (e.g., a SULF construct), a wide variety of which are known in the art and need not be elaborated upon herein. Vectors include, but are not limited to, plasmids; cosmids; viral vectors; artificial chromosomes (YAC's, BAC's, etc.); mini-chromosomes; and the like. Vectors are amply described in numerous publications well known to those in the art, including, e.g., Short Protocols in Molecular Biology, (1999) F. Ausubel, et al., eds., Wiley & Sons. Vectors may provide for expression of the subject nucleic acids, may provide for propagating the subject nucleic acids, or both.

The subject genes are isolated and obtained in substantial purity, generally as other than an intact chromosome. Usually, the DNA will be obtained substantially free of other nucleic acid sequences that do not include a sequence or fragment thereof of the subject genes, generally being at least about 50%, usually at least about 90% pure and are typically “recombinant”, i.e. flanked by one or more nucleotides with which it is not normally associated on a naturally occurring chromosome.

Preparation of the Subject Polypeptides

In addition to the plurality of uses described in greater detail in following sections, the subject nucleic acid compositions find use in the preparation of all or a portion of the sulfatase polypeptides of the subject invention, as described above. For expression, an expression cassette may be employed. The expression vector will provide a transcriptional and translational initiation region, which may be inducible or constitutive, where the coding region is operably linked under the transcriptional control of the transcriptional initiation region, and a transcriptional and translational termination region. These control regions may be native to a gene encoding the subject peptides, or may be derived from exogenous sources.

Expression vectors generally have convenient restriction sites located near the promoter sequence to provide for the insertion of nucleic acid sequences encoding heterologous proteins. A selectable marker operative in the expression host may be present. Expression vectors may be used for the production of fusion proteins, where the exogenous fusion peptide provides additional functionality, i.e. increased protein synthesis, stability, reactivity with defined antisera, an enzyme marker, e.g. β-galactosidase, etc.

Expression cassettes may be prepared comprising a transcription initiation region, the gene or fragment thereof, and a transcriptional termination region. Of particular interest is the use of sequences that allow for the expression of functional epitopes or domains, usually at least about 8 amino acids in length, more usually at least about 15 amino acids in length, to about 25 amino acids, or any of the above-described fragment, and up to the complete open reading frame of the gene. After introduction of the DNA, the cells containing the construct may be selected by means of a selectable marker, the cells expanded and then used for expression.

Proteins and polypeptides may be expressed in prokaryotes or eukaryotes in accordance with conventional ways, depending upon the purpose for expression. For large scale production of the protein, a unicellular organism, such as E. coli, B. subtilis, S. cerevisiae, insect cells in combination with baculovirus vectors, or cells of a higher organism such as vertebrates, particularly mammals, e.g. COS 7 cells, may be used as the expression host cells. In some situations, it is desirable to express the gene in eukaryotic cells, where the encoded protein will benefit from native folding and post-translational modifications. Small peptides can also be synthesized in the laboratory. Polypeptides that are subsets of the complete sequences of the subject proteins may be used to identify and investigate parts of the protein important for function.

Specific expression systems of interest include bacterial, yeast, insect cell and mammalian cell derived expression systems. Representative systems from each of these categories is are provided below:

Bacteria. Expression systems in bacteria include those described in Chang et al., Nature (1978) 275:615; Goeddel et al., Nature (1979) 281:544; Goeddel et al., Nucleic Acids Res. (1980) 8:4057; EP 0 036,776; U.S. Pat. No. 4,551,433; DeBoer et al., Proc. Natl. Acad. Sci. (USA) (1983) 80:21-25; and Siebenlist et al., Cell (1980) 20:269.

Yeast. Expression systems in yeast include those described in Hinnen et al., Proc. Natl. Acad. Sci. (USA) (1978) 75:1929; Ito et al., J. Bacteriol. (1983) 153:163; Kurtz et al., Mol. Cell. Biol. (1986) 6:142; Kunze et al., J. Basic Microbiol. (1985) 25:141; Gleeson et al., J. Gen. Microbiol. (1986) 132:3459; Roggenkamp et al., Mol. Gen. Genet. (1986) 202:302; Das et al., J. Bacteriol. (1984) 158:1165; De Louvencourt et al., J. Bacteriol. (1983) 154:737; Van den Berg et al., Bio/Technology (1990) 8:135; Kunze et al., J. Basic Microbiol. (1985) 25:141; Cregg et al., Mol. Cell. Biol. (1985) 5:3376; U.S. Pat. Nos. 4,837,148 and 4,929,555; Beach and Nurse, Nature (1981) 300:706; Davidow et al., Curr. Genet. (1985) 10:380; Gaillardin et al., Curr. Genet. (1985) 10:49; Ballance et al., Biochem. Biophys. Res. Commun. (1983) 112:284-289; Tilburn et al., Gene (1983) 26:205-221; Yelton et al., Proc. Natl. Acad. Sci. (USA) (1984) 81:1470-1474; Kelly and Hynes, EMBO J. (1985) 4:475479; EP 0 244,234; and WO 91/00357.

Insect Cells. Expression of heterologous genes in insects is accomplished as described in U.S. Pat. No. 4,745,051; Friesen et al., “The Regulation of Baculovirus Gene Expression”, in: The Molecular Biology of Baculoviruses (1986) (W. Doerfler, ed.); EP 0 127,839; EP 0 155,476; and Vlak et al., J. Gen. Virol. (1988) 69:765-776; Miller et al., Ann. Rev. Microbiol. (1988) 42:177; Carbonell et al., Gene (1988) 73:409; Maeda et al., Nature (1985) 315:592-594; Lebacq-Verheyden et al., Mol. Cell Biol. (1988) 8:3129; Smith et al., Proc. Natl. Acad. Sci. (USA) (1985) 82:8844; Miyajima et al., Gene (1987) 58:273; and Martin et al., DNA (1988) 7:99. Numerous baculoviral strains and variants and corresponding permissive insect host cells from hosts are described in Luckow et al., Bio/Technology (1988) 6:47-55, Miller et al., Generic Engineering (1986) 8:277-279, and Maeda et al., Nature (1985) 315:592-594.

Mammalian Cells. Mammalian expression is accomplished as described in Dijkema et al., EMBO J. (1985) 4:761, Gorman et al., Proc. Natl. Acad. Sci. (USA) (1982) 79:6777, Boshart et al., Cell (1985) 41:521 and U.S. Pat. No. 4,399,216. Other features of mammalian expression are facilitated as described in Ham and Wallace, Meth. Enz. (1979) 58:44, Barnes and Sato, Anal. Biochem. (1980) 102:255, U.S. Pat. Nos. 4,767,704, 4,657,866, 4,927,762, 4,560,655, WO 90/103430, WO 87/00195, and U.S. RE 30,985.

When any of the above host cells, or other appropriate host cells or organisms, are used to replicate and/or express the polynucleotides or nucleic acids of the invention, the resulting replicated nucleic acid, RNA, expressed protein or polypeptide, is within the scope of the invention as a product of the host cell or organism. The product is recovered by any appropriate means known in the art.

Once the gene corresponding to a selected polynucleotide is identified, its expression can be regulated in the cell to which the gene is native. For example, an endogenous gene of a cell can be regulated by an exogenous regulatory sequence inserted into the genome of the cell at location sufficient to at least enhance expressed of the gene in the cell. The regulatory sequence may be designed to integrate into the genome via homologous recombination, as disclosed in U.S. Pat. Nos. 5,641,670 and 5,733,761, the disclosures of which are herein incorporated by reference, or may be designed to integrate into the genome via non-homologous recombination, as described in WO 99/15650, the disclosure of which is herein incorporated by reference. As such, also encompassed in the subject invention is the production of the subject proteins without manipulation of the encoding nucleic acid itself, but instead through integration of a regulatory sequence into the genome of cell that already includes a gene encoding the desired protein, as described in the above incorporated patent documents.

The subject proteins and polypeptides may be obtained from naturally occurring sources or synthetically produced. For example, the proteins may be derived from biological sources which express the proteins. The subject proteins may also be derived from synthetic means, e.g. by expressing a recombinant gene encoding protein of interest in a suitable host, as described in greater detail infra. Any convenient protein purification procedures may be employed, where suitable protein purification methodologies are described in Guide to Protein Purification, (Deuthser ed.) (Academic Press, 1990). For example, a lysate may prepared from the original source, (e.g. a cell expressing endogenous SULF1 or SULF2, or a cell comprising the expression vector expressing the subject polypeptide(s)), and purified using HPLC, exclusion chromatography, gel electrophoresis, affinity chromatography, and the like.

Compositions

The present invention further provides compositions, including pharmaceutical compositions, comprising the polypeptides, polynucleotides, antibodies, recombinant vectors, and host cells of the invention. These compositions may include a buffer, which is selected according to the desired use of the polypeptide, antibody, polynucleotide, recombinant vector, or host cell, and may also include other substances appropriate to the intended use. Those skilled in the art can readily select an appropriate buffer, a wide variety of which are known in the art, suitable for an intended use. In some instances, the composition can comprise a pharmaceutically acceptable excipient, a variety of which are known in the art and need not be discussed in detail herein. Pharmaceutically acceptable excipients have been amply described in a variety of publications, including, for example, A. Gennaro (1995) “Remington: The Science and Practice of Pharmacy”, 19th edition, Lippincott, Williams, & Wilkins.

Antibodies Specific for a Sulfatase of the Invention

The invention provides antibodies that are specific for a subject sulfatase. Suitable antibodies are obtained by immunizing a host animal with peptides comprising all or a portion of the target protein. Suitable host animals include mouse, rat sheep, goat, hamster, rabbit, etc. The origin of the protein immunogen may be mouse, human, rat, monkey etc. The host animal will generally be a different species than the immunogen, e.g. human protein used to immunize mice, etc.

The immunogen may comprise the complete protein, or fragments and derivatives thereof. Preferred immunogens comprise all or a part of one of the subject proteins, where these residues contain the post-translation modifications, such as glycosylation, found on the native target protein. Immunogens comprising the extracellular domain are produced in a variety of ways known in the art, e.g. expression of cloned genes using conventional recombinant methods, isolation from tumor cell culture supernatants, etc.

For preparation of polyclonal antibodies, the first step is immunization of the host animal with the target protein, where the target protein will preferably be in substantially pure form, comprising less than about 1% contaminant. The immunogen may comprise the complete target protein, fragments or derivatives thereof. To increase the immune response of the host animal, the target protein may be combined with an adjuvant, where suitable adjuvants include alum, dextran, sulfate, large polymeric anions, oil & water emulsions, e.g. Freund's adjuvant, Freund's complete adjuvant, and the like. The target protein may also be conjugated to synthetic carrier proteins or synthetic antigens. A variety of hosts may be immunized to produce the polyclonal antibodies. Such hosts include rabbits, guinea pigs, rodents, e.g. mice, rats, sheep, goats, and the like. The target protein is administered to the host, usually intradermally, with an initial dosage followed by one or more, usually at least two, additional booster dosages. Following immunization, the blood from the host will be collected, followed by separation of the serum from the blood cells. The Ig present in the resultant antiserum may be further fractionated using known methods, such as ammonium salt fractionation, DEAE chromatography, and the like.

Monoclonal antibodies are produced by conventional techniques. Generally, the spleen and/or lymph nodes of an immunized host animal provide a source of plasma cells. The plasma cells are immortalized by fusion with myeloma cells to produce hybridoma cells. Culture supernatant from individual hybridomas is screened using standard techniques to identify those producing antibodies with the desired specificity. Suitable animals for production of monoclonal antibodies to the human protein include mouse, rat, hamster, etc. To raise antibodies against the mouse protein, the animal will generally be a hamster, guinea pig, rabbit, etc. The antibody may be purified from the hybridoma cell supernatants or ascites fluid by conventional techniques, e.g. affinity chromatography using protein according to the subject invention bound to an insoluble support, protein A sepharose, etc.

The antibody may be produced as a single chain, instead of the normal multimeric structure. Single chain antibodies are described in Jost et al. (1994) J.B.C. 269:26267-73, and others. DNA sequences encoding the variable region of the heavy chain and the variable region of the light chain are ligated to a spacer encoding at least about 4 amino acids of small neutral amino acids, including glycine and/or serine. The protein encoded by this fusion allows assembly of a functional variable region that retains the specificity and affinity of the original antibody.

For in vivo use, particularly for injection into humans, it is desirable to decrease the antigenicity of the antibody. An immune response of a recipient against the blocking agent will potentially decrease the period of time that the therapy is effective. Methods of humanizing antibodies are known in the art. The humanized antibody may be the product of an animal having transgenic human immunoglobulin constant region genes (see for example International Patent Applications WO 90/10077 and WO 90/04036). Alternatively, the antibody of interest may be engineered by recombinant DNA techniques to substitute the CH1, CH2, CH3, hinge domains, and/or the framework domain with the corresponding human sequence (see WO 92/02190).

The use of Ig cDNA for construction of chimeric immunoglobulin genes is known in the art (Liu et al. (1987) P.N.A.S. 84:3439 and (1987) J. Immunol. 139:3521). mRNA is isolated from a hybridoma or other cell producing the antibody and used to produce cDNA. The cDNA of interest may be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant regions genes may be found in Kabat et al. (1991) Sequences of Proteins of Immunological Interest, N.I.H. publication no. 91-3242. Human C region genes are readily available from known clones. The choice of isotype will be guided by the desired effector functions, such as complement fixation, or activity in antibody-dependent cellular cytotoxicity. Preferred isotypes are IgG1, IgG3 and IgG4. Either of the human light chain constant regions, kappa or lambda, may be used. The chimeric, humanized antibody is then expressed by conventional methods.

In yet other embodiments, the antibodies may be fully human antibodies. For example, xenogeneic antibodies which are identical to human antibodies may be employed. By xenogenic human antibodies is meant antibodies that are the same has human antibodies, i.e. they are fully human antibodies, with exception that they are produced using a non-human host which has been genetically engineered to express human antibodies. See e.g. WO 98/50433; WO 98,24893 and WO 99/53049, the disclosures of which are herein incorporated by reference.

Antibody fragments, such as Fv, F(ab′)₂ and Fab may be prepared by cleavage of the intact protein, e.g. by protease or chemical cleavage. Alternatively, a truncated gene is designed. For example, a chimeric gene encoding a portion of the F(ab′)₂ fragment would include DNA sequences encoding the CH1 domain and hinge region of the H chain, followed by a translational stop codon to yield the truncated molecule.

Consensus sequences of H and L J regions may be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

Expression vectors include plasmids, retroviruses, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody may be joined to any strong promoter, including retroviral LTRs, e.g. SV-40 early promoter, (Okayama et al. (1983) Mol. Cell. Bio. 3:280), Rous sarcoma virus LTR (Gorman et al. (1982) P.N.A.S. 79:6777), and moloney murine leukemia virus LTR (Grosschedl et al. (1985) Cell 41:885); native Ig promoters, etc.

Uses of the Subject Polypeptide and Nucleic Acid Compositions

The subject polypeptide and nucleic acid compositions find use in a variety of different applications, including research, diagnostic, and therapeutic agent screening/discovery/preparation applications, as well as therapeutic compositions.

General Applications

The subject nucleic acid compositions find use in a variety of different applications. Applications of interest include: the identification of homologs of the subject sulfatases; as a source of novel promoter elements; the identification of expression regulatory factors; as probes and primers in hybridization applications, e.g. polymerase chain reaction (PCR); the identification of expression patterns in biological specimens; the preparation of cell or animal models for function of the subject sulfatases; the preparation of in vitro models for function of the subject sulfatases; etc.

Homologs are identified by any of a number of methods. A fragment of the provided cDNA may be used as a hybridization probe against a cDNA library from the target organism of interest, where low stringency conditions are used. The probe may be a large fragment, or one or more short degenerate primers. Nucleic acids having sequence similarity are detected by hybridization under low stringency conditions, for example, at 50° C. and 6×SSC (0.9 M sodium chloride/0.09 M sodium citrate) and remain bound when subjected to washing at 55° C. in 1×SSC (0.15 M sodium chloride/0.015 M sodium citrate). Sequence identity may be determined by hybridization under stringent conditions, for example, at 50° C. or higher and 0.1×SSC (15 mM sodium chloride/01.5 mM sodium citrate). Nucleic acids having a region of substantial identity to the provided nucleic acid sequences, e.g. allelic variants, genetically altered versions of the gene, etc., bind to the provided sequences under stringent hybridization conditions. By using probes, particularly labeled probes of DNA sequences, one can isolate homologous or related genes.

The sequence of the 5′ flanking region may be utilized for promoter elements, including enhancer binding sites, that provide for developmental regulation in tissues where the subject genes are expressed. The tissue specific expression is useful for determining the pattern of expression, and for providing promoters that mimic the native pattern of expression. Naturally occurring polymorphisms in the promoter region are useful for determining natural variations in expression, particularly those that may be associated with disease.

Alternatively, mutations may be introduced into the promoter region to determine the effect of altering expression in experimentally defined systems. Methods for the identification of specific DNA motifs involved in the binding of transcriptional factors are known in the art, e.g. sequence similarity to known binding motifs, gel retardation studies, etc. For examples, see Blackwell et al. (1995), Mol. Med. 1:194-205; Mortlock et al. (1996), Genome Res. 6:327-33; and Joulin and Richard-Foy (1995), Eur. J. Biochem. 232:620-626.

The regulatory sequences may be used to identify cis acting sequences required for transcriptional or translational regulation of expression, especially in different tissues or stages of development, and to identify cis acting sequences and trans-acting factors that regulate or mediate expression. Such transcription or translational control regions may be operably linked to a gene in order to promote expression of wild type or proteins of interest in cultured cells, or in embryonic, fetal or adult tissues, and for gene therapy.

Small DNA fragments are useful as primers for PCR, hybridization screening probes, etc. Larger DNA fragments, i.e. greater than 100 nt are useful for production of the encoded polypeptide, as described in the previous section. For use in amplification reactions, such as PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other.

The DNA may also be used to identify expression of the gene in a biological specimen. The manner in which one probes cells for the presence of particular nucleotide sequences, as genomic DNA or RNA, is well established in the literature. Briefly, DNA or mRNA is isolated from a cell sample. The mRNA may be amplified by RT-PCR, using reverse transcriptase to form a complementary DNA strand, followed by polymerase chain reaction amplification using primers specific for the subject DNA sequences. Alternatively, the mRNA sample is separated by gel electrophoresis, transferred to a suitable support, e.g. nitrocellulose, nylon, etc., and then probed with a fragment of the subject DNA as a probe. Other techniques, such as oligonucleotide ligation assays, in situ hybridizations, and hybridization to DNA probes arrayed on a solid chip may also find use. Detection of mRNA hybridizing to the subject sequence is indicative of gene expression in the sample.

The sequence of a gene according to the subject invention, including flanking promoter regions and coding regions, may be mutated in various ways known in the art to generate targeted changes in promoter strength, sequence of the encoded protein, etc. The DNA sequence or protein product of such a mutation will usually be substantially similar to the sequences provided herein, i.e. will differ by at least one nucleotide or amino acid, respectively, and may differ by at least two but not more than about ten nucleotides or amino acids. The sequence changes may be substitutions, insertions, deletions, or a combination thereof. Deletions may further include larger changes, such as deletions of a domain or exon. Other modifications of interest include epitope tagging, e.g. with the FLAG system, HA, etc. For studies of subcellular localization, fusion proteins with green fluorescent proteins (GFP) may be used.

Techniques for in vitro mutagenesis of cloned genes are known. Examples of protocols for site specific mutagenesis may be found in Gustin et al. (1993), Biotechniques 14:22; Barany (1985), Gene 37:111-23; Colicelli et al. (1985), Mol. Gen. Genet. 199:537-9; and Prentki et al. (1984), Gene 29:303-13. Methods for site specific mutagenesis can be found in Sambrook et al, Molecular Cloning: A Laboratory Manual, CSH Press 1989, pp. 15.3-15.108; Weiner et al. (1993), Gene 126:35-41; Sayers et al. (1992), Biotechniques 13:592-6; Jones and Winistorfer (1992), Biotechniques 12:528-30; Barton et al. (1990), Nucleic Acids Res 18:7349-55; Marotti and Tomich (1989), Gene Anal. Tech. 6:67-70; and Zhu (1989), Anal Biochem 177:120-4. Such mutated genes may be used to study structure-function relationships of the subject proteins, or to alter properties of the protein that affect its function or regulation.

The subject nucleic acids can be used to generate transgenic, non-human animals or site-specific gene modifications in cell lines. Thus, in some embodiments, the invention provides a non-human transgenic animal comprising, as a transgene integrated into the genome of the animal, a nucleic acid molecule comprising a sequence encoding a subject sulfatase in operable linkage with a promoter, such that the sulfatase-encoding nucleic acid molecule is expressed in a cell of the animal. Transgenic animals may be made through homologous recombination, where the endogenous locus is altered. Alternatively, a nucleic acid construct is randomly integrated into the genome. Vectors for stable integration include plasmids, retroviruses and other animal viruses, YACs, and the like.

The modified cells or animals are useful in the study of gene function and regulation. For example, a series of small deletions and/or substitutions may be made in the host's native gene to determine the role of different exons in oncogenesis, signal transduction, etc. Of interest is the use of genes to construct transgenic animal models for cancer, where expression of the subject protein is specifically reduced or absent. Specific constructs of interest include anti-sense constructs, which will block expression, expression of dominant negative mutations, and over-expression of genes. Where a sequence is introduced, the introduced sequence may be either a complete or partial sequence of a gene native to the host, or may be a complete or partial sequence that is exogenous to the host animal, e.g., a human sequence of the subject invention. A detectable marker, such as lac Z may be introduced into the locus, where upregulation of expression will result in an easily detected change in phenotype.

One may also provide for expression of the gene, e.g. the SULF1 or SULF2 gene, or variants thereof in cells or tissues where it is not normally expressed, at levels not normally present in such cells or tissues, or at abnormal times of development. One may also generate host cells (including host cells in transgenic animals) that comprise a heterologous nucleic acid molecule which encodes a polypeptide which functions to modulate expression of an endogenous SULF1 or SULF2 promoter or other transcriptional regulatory region.

DNA constructs for homologous recombination will comprise at least a portion of the human gene or of a gene native to the species of the host animal, wherein the gene has the desired genetic modification(s), and includes regions of homology to the target locus. DNA constructs for random integration need not include regions of homology to mediate recombination. Conveniently, markers for positive and negative selection are included. Methods for generating cells having targeted gene modifications through homologous recombination are known in the art. For various techniques for transfecting mammalian cells, see Keown et al. (1990), Meth. Enzymol. 185:527-537.

For embryonic stem (ES) cells, an ES cell line may be employed, or embryonic cells may be obtained freshly from a host, e.g. mouse, rat, guinea pig, etc. Such cells are grown on an appropriate fibroblast-feeder layer or grown in the presence of leukemia inhibiting factor (LIF). When ES or embryonic cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting offspring screened for the construct. By providing for a different phenotype of the blastocyst and the genetically modified cells, chimeric progeny can be readily detected.

The chimeric animals are screened for the presence of the modified gene and males and females having the modification are mated to produce homozygous progeny. If the gene alterations cause lethality at some point in development, tissues or organs can be maintained as allogeneic or congenic grafts or transplants, or in in vitro culture. The transgenic animals may be any non-human mammal, such as laboratory animals, domestic animals, etc. The transgenic animals may be used in functional studies, drug screening, etc., e.g. to determine the effect of a candidate drug on SULF1 or SULF2 activity.

Diagnostic Applications

Also provided are methods of diagnosing disease states based on observed levels and/or activity of the subject sulfatase(s) and/or the level of a subject sulfatase polynucleotide in a biological sample of interest. Samples, as used herein, include biological fluids such as blood, cerebrospinal fluid, tears, saliva, lymph, dialysis fluid, breast ductal lavage fluid, semen and the like; cells; organ or tissue culture derived fluids; tumor biopsy samples; stool samples; and fluids extracted from physiological tissues. Also included in the term are derivatives and fractions of such fluids. The cells may be dissociated, in the case of solid tissues, or tissue sections may be analyzed. Alternatively a lysate of the cells may be prepared.

Detection methods of the invention may be qualitative or quantitative. Thus, as used herein, the terms “detection,” “determination,” and the like, refer to both qualitative and quantitative determinations, and include “measuring.”

Detection methods of the present invention include methods for detecting sulfatase polypeptide in a biological sample, methods for detecting sulfatase mRNA in a biological sample, and methods for detecting sulfatase enzymatic activity in a biological sample.

In some embodiments, the detection methods provide for detection of cancerous cells in a biological sample (e.g., a tissue biopsy). As described in the Examples, huSULF-1 mRNA levels are elevated in particular cancers, e.g., pancreatic cancer and prostate cancer; and huSULF-2 mRNA levels are elevated in breast cancer. Thus, detection of an mRNA encoding huSULF-1 or huSULF-2 at an elevated level compared to normal (non-cancerous) tissue, provides for detection of cancerous tissue in a biological sample.

Detection Kits

The detection methods can be provided as part of a kit. Thus, the invention further provides kits for detecting the presence and/or a level of sulfatase polypeptide or sulfatase polynucleotide in a biological sample. Procedures using these kits can be performed by clinical laboratories, experimental laboratories, medical practitioners, or private individuals. The kits of the invention for detecting a sulfatase polypeptide comprise a moiety that specifically binds sulfatase, including, but not limited to, a sulfatase-specific antibody. The kits of the invention for detecting a sulfatase polynucleotide comprise a moiety that specifically hybridizes to a sulfatase polynucleotide.

In some embodiments, a kit of the invention for detecting a sulfatase polynucleotide, such as an mRNA encoding a subject sulfatase, comprises a pair of nucleic acids that function as “forward” and “reverse” primers that specifically amplify a cDNA copy of a subject sulfatase-encoding mRNA. The “forward” and “reverse” primers are provided in the kit as a pair of isolated nucleic acid molecules, each from about 10 to 200 nucleotides in length, the first nucleic acid molecule of the pair comprising a sequence of at least 10 contiguous nucleotides having 100% sequence identity to the nucleic acid sequence set forth in any one of SEQ ID NO:02, 05, or 14, and the second nucleic acid molecule of the pair comprising a sequence of at least 10 contiguous nucleotides having 100% sequence identity to the reverse complement of the nucleic acid sequence set forth in any one of SEQ ID NO:02, 05, or 14, wherein the sequence of the second nucleic acid molecule is located 3′ of the nucleic acid sequence of the first nucleic acid molecule in any one of SEQ ID NO:02, 05, or 14. The primer nucleic acids are prepared using any known method, e.g., automated synthesis, and the like.

The invention provides a kit comprising a pair of nucleic acids as described above. The nucleic acids are present in a suitable storage medium, e.g., buffered solution, typically in a suitable container. The kit includes the pair of nucleic acids, and may further include a buffer; reagents for polymerase chain reaction (e.g., deoxynucleotide triphosphates (dATP, dTTP, dCTP, and dGTP), a thermostable DNA polymerase, a buffer suitable for polymerase chain reaction, a solution containing Mg²⁺ ions (e.g., MgCl₂), and other components well known to those skilled in the art for carrying out a polymerase chain reaction). The kit may further include instructions for use of the kit, which instructions may be provided in a variety of forms, e.g., as printed information, on a compact disc, and the like. The kit may further include reagents necessary for extraction of DNA from a biological sample (e.g., biopsy sample, blood, and the like) from an individual, and reagents for generating a cDNA copy of an mRNA. The kits are useful in diagnostic applications, as described in more detail below. The pair of isolated nucleic acid molecules serve as primers in an amplification reaction (e.g., a polymerase chain reaction).

In some embodiments, the first and/or the second nucleic acid molecules comprises a detectable label. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affnity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The kit may optionally provide additional components that are useful in the procedure, including, but not limited to, buffers, developing reagents, labels, reacting surfaces, means for detections, control samples, standards, instructions, and interpretive information.

Where the kit provides for detection of a subject sulfatase polypeptide, the kit includes one or more antibodies specific for the subject sulfatase. In some embodiments, the antibody specific for the subject sulfatase is detectably labeled. In other embodiments, the antibody specific for the subject sulfatase is not labeled; instead, a second, detectably-labeled antibody is provided that binds to the antibody specific for a subject sulfatase (the “first” antibody). The kit may further include blocking reagents, buffers, and reagents for developing and/or detecting the detectable marker. The kit may further include instructions for use, controls, and interpretive information.

Where the kit provides for detecting enzymatic activity of a subject sulfatase, the kit includes a substrate that provides for a detectable product when acted upon by a subject sulfatase. Suitable substrates are discussed in detail below. One non-limiting example of a suitable substrate is 4-methylumbelliferyl-sulfate. The kit may further include reagents necessary for detectable marker development and detection. The kit may further include instructions for use, controls, and interpretive information.

Methods of Detecting a Sulfatase Polypeptide in a Biological Sample

The present invention further provides methods for detecting the presence and/or measuring a level of a sulfatase polypeptide in a biological sample, using a sulfatase-specific antibody. The methods generally comprise:
a) contacting the sample with an antibody specific for a sulfatase polypeptide; and
b) detecting binding between the antibody and molecules of the sample.

Detection of specific binding of the sulfatase-specific antibody, when compared to a suitable control, is an indication that sulfatase polypeptides are present in the sample. Suitable controls include a sample known not to contain a sulfatase polypeptide; and a sample contacted with an antibody not specific for sulfatase, e.g., an anti-idiotype antibody. A variety of methods to detect specific antibody-antigen interactions are known in the art and can be used in the method, including, but not limited to, standard immunohistological methods, immunoprecipitation, an enzyme immunoassay, and a radioimmunoassay. In general, the sulfatase-specific antibody will be detectably labeled, either directly or indirectly. Direct labels include radioisotopes; enzymes whose products are detectable (e.g., luciferase, β-galactosidase, and the like); fluorescent labels (e.g., fluorescein isothiocyanate, rhodamine, phycoerythrin, and the like); fluorescence emitting metals, e.g., ¹⁵²Eu, or others of the lanthanide series, attached to the antibody through metal chelating groups such as EDTA; chemiluminescent compounds, e.g., luminol, isoluminol, acridinium salts, and the like; bioluminescent compounds, e.g., luciferin, aequorin (green fluorescent protein), and the like.

The antibody may be attached (coupled) to an insoluble support, such as a polystyrene plate or a bead. Indirect labels include second antibodies specific for sulfatase-specific antibodies, wherein the second antibody is labeled as described above; and members of specific binding pairs, e.g., biotin-avidin, and the like. The biological sample may be brought into contact with an immobilized on a solid support or carrier, such as nitrocellulose, that is capable of immobilizing cells, cell particles, or soluble proteins. The support may then be washed with suitable buffers, followed by contacting with a detectably-labeled sulfatase-specific antibody. Detection methods are known in the art and will be chosen as appropriate to the signal emitted by the detectable label. Detection is generally accomplished in comparison to suitable controls, and to appropriate standards.

Methods of Detecting Enzymatic Activity of a Subject Sulfatase in a Biological Sample

The present invention further provides methods for detecting the presence and/or levels of enzymatic activity of a subject sulfatase in a biological sample. The methods generally involve:
a) contacting the sample with a substrate that yields a detectable product upon being acted upon by a subject sulfatase; and
b) detecting a product of the enzymatic reaction.

Any sulfated compound that, upon cleavage of the sulfate group by the sulfatase activity, results in a change in absorption, fluorescence or other physical property amenable to detection, is suitable for use in a subject assay. Suitable substrates include, but are not limited to, 4-methylumbelliferyl sulfate; p-nitrophenyl sulfate; 4-methylumbelliferyl-α-D-N-acetylglucosamide-6-sulfate or 4-methylumbelliferyl-glucosamine-6-sulfate or conjugates containing these derivatives; any sulfated sugar or assembly of sugars related to heparan sulfate, including fragments of heparin or heparan sulfate; and any sulfated compound in which the sulfate is radiolabeled.

Methods of Detecting a Sulfatase mRNA in a Biological Sample

The present invention further provides methods for detecting the presence of sulfatase mRNA in a biological sample. The methods can be used, for example, to assess whether a test compound affects sulfatase gene expression, directly or indirectly.

The methods generally comprise:
a) contacting the sample with a sulfatase polynucleotide of the invention under conditions which allow hybridization; and
b) detecting hybridization, if any.

Detection of hybridization, when compared to a suitable control, is an indication of the presence in the sample of a sulfatase polynucleotide. Appropriate controls include, for example, a sample which is known not to contain sulfatase mRNA, and use of a labelled polynucleotide of the same “sense” as a sulfatase mRNA. Conditions which allow hybridization are known in the art, and have been described in more detail above. Detection can be accomplished by any known method, including, but not limited to, in situ hybridization, PCR, RT-PCR, and “Northern” or RNA blotting, or combinations of such techniques, using a suitably labelled sulfatase polynucleotide. A variety of labels and labelling methods for polynucleotides are known in the art and can be used in the assay methods of the invention. Specific hybridization can be determined by comparison to appropriate controls.

In some embodiments, the methods involve generating a cDNA copy of an mRNA molecule in a biological sample, and amplifying the cDNA using a pair of isolated nucleic acid molecules that serve as forward and reverse primers in an amplification reaction (e.g., a polymerase chain reaction). Each of the nucleic acid molecules in the pair of nuclei acid molecules is from about 10 to 200 nucleotides in length, the first nucleic acid molecule of the pair comprising a sequence of at least 10 contiguous nucleotides having 100% sequence identity to the nucleic acid sequence set forth in any one of SEQ ID NO:02, 05, or 14, and the second nucleic acid molecule of the pair comprising a sequence of at least 10 contiguous nucleotides having 100% sequence identity to the reverse complement of the nucleic acid sequence set forth in any one of SEQ ID NO:02, 05, or 14, wherein the sequence of the second nucleic acid molecule is located 3′ of the nucleic acid sequence of the first nucleic acid molecule in any one of SEQ ID NO:02, 05, or 14. The primer nucleic acids are prepared using any known method, e.g., automated synthesis, and the like. The primer pairs are chosen such that they specifically amplify a cDNA copy of an mRNA encoding a subject sulfatase.

Methods using PCR amplification can be performed on the DNA from a single cell, although it is convenient to use at least about 10⁵ cells. The use of the polymerase chain reaction is described in Saiki et al. (1985) Science 239:487, and a review of current techniques may be found in Sambrook, et al. Molecular Cloning: A Laboratory Manual, CSH Press 1989, pp. 14.2-14.33. A detectable label may be included in the amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, 3H; etc. The label may be a two stage system, where the amplified DNA is conjugated to biotin, haptens, etc. having a high affnity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

A number of methods are available for determining the expression level of a gene or protein in a particular sample. Diagnosis may be performed by a number of methods to determine the absence or presence or altered amounts of normal or abnormal sulfatase in a patient sample. For example, detection may utilize staining of cells or histological sections with labeled antibodies, performed in accordance with conventional methods. Cells are permeabilized to stain cytoplasmic molecules. The antibodies of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Alternatively, the secondary antibody conjugated to a fluorescent compound, e.g. fluorescein, rhodamine, Texas red, etc. Final detection uses a substrate that undergoes a color change in the presence of the peroxidase. The absence or presence of antibody binding may be determined by various methods, including flow cytometry of dissociated cells, microscopy, radiography, scintillation counting, etc.

Alternatively, one may focus on the expression of the subject sulfatase genes. Biochemical studies may be performed to determine whether a sequence polymorphism in a coding region or control regions is associated with disease. Disease associated polymorphisms may include deletion or truncation of the gene, mutations that alter expression level, that affect the activity of the protein, etc.

Changes in the promoter or enhancer sequence that may affect expression levels of the subject genes can be compared to expression levels of the normal allele by various methods known in the art. Methods for determining promoter or enhancer strength include quantitation of the expressed natural protein; insertion of the variant control element into a vector with a reporter gene such as β-galactosidase, luciferase, chloramphenicol acetyltransferase, etc. that provides for convenient quantitation; and the like.

A number of methods are available for analyzing nucleic acids for the presence of a specific sequence, e.g. a disease associated polymorphism. Where large amounts of DNA are available, genomic DNA is used directly. Alternatively, the region of interest is cloned into a suitable vector and grown in sufficient quantity for analysis. Cells that express the gene may be used as a source of mRNA, which may be assayed directly or reverse transcribed into cDNA for analysis. The nucleic acid may be amplified by conventional techniques, such as the polymerase chain reaction (PCR), to provide sufficient amounts for analysis. The use of the polymerase chain reaction is described in Saiki, et al. (1985), Science 239:487, and a review of techniques may be found in Sambrook, et al. Molecular Cloning: A Laboratory Manual, CSH Press 1989, pp. 14.2-14.33. Alternatively, various methods are known in the art that utilize oligonucleotide ligation as a means of detecting polymorphisms, for examples see Riley et al. (1990), Nucl. Acids Res. 18:2887-2890; and Delahunty et al. (1996), Am. J. Hum. Genet. 58:1239-1246.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified or cloned fragment, is analyzed by one of a number of methods known in the art. The nucleic acid may be sequenced by dideoxy or other methods, and the sequence of bases compared to a wild-type sequence. Hybridization with the variant sequence may also be used to determine its presence, by Southern blots, dot blots, etc. The hybridization pattern of a control and variant sequence to an array of oligonucleotide probes immobilized on a solid support, as described in U.S. Pat. No. 5,445,934, or in WO 95/35505, may also be used as a means of detecting the presence of variant sequences. Single strand conformational polymorphism (SSCP) analysis, denaturing gradient gel electrophoresis (DGGE), and heteroduplex analysis in gel matrices are used to detect conformational changes created by DNA sequence variation as alterations in electrophoretic mobility. Alternatively, where a polymorphism creates or destroys a recognition site for a restriction endonuclease; the sample is digested with that endonuclease, and the products size fractionated to determine whether the fragment was digested. Fractionation is performed by gel or capillary electrophoresis, particularly acrylamide or agarose gels.

Screening for mutations in the gene may be based on the functional or antigenic characteristics of the protein. Protein truncation assays are useful in detecting deletions that may affect the biological activity of the protein. Various immunoassays designed to detect polymorphisms in proteins may be used in screening. Where many diverse genetic mutations lead to a particular disease phenotype, functional protein assays have proven to be effective screening tools. The activity of the encoded protein may be determined by comparison with the wild-type protein.

Diagnostic methods of the subject invention in which the level of expression is of interest will typically involve comparison of the nucleic acid abundance of a sample of interest with that of a control value to determine any relative differences, where the difference may be measured qualitatively and/or quantitatively, which differences are then related to the presence or absence of an abnormal expression pattern. A variety of different methods for determining the nucleic acid abundance in a sample are known to those of skill in the art, where particular methods of interest include those described in: Pietu et al., Genome Res. (June 1996) 6: 492-503; Zhao et al., Gene (Apr. 24, 1995) 156: 207-213; Soares, Curr. Opin. Biotechnol. (October 1997) 8: 542-546; Raval, J. Pharmacol Toxicol Methods (November 1994) 32: 125-127; Chalifour et al., Anal. Biochem (Feb. 1, 1994) 216: 299-304; Stolz & Tuan, Mol. Biotechnol. (December 19960 6: 225-230; Hong et al., Bioscience Reports (1982) 2: 907; and McGraw, Anal. Biochem. (1984) 143: 298. Also of interest are the methods disclosed in WO 97/27317, the disclosure of which is herein incorporated by reference.

Screening Assays

The present invention provides screening methods for identifying agents which modulate sulfatase enzyme activity, methods for identifying agents which modulate a level of a subject sulfatase polypeptide in a cell; and methods for identifying agents which modulate a level of a subject sulfatase mRNA in a cell; and methods for identifying agents that modulate release of a subject sulfatase from a eukaryotic cell. In some embodiments, the assay is a cell-free assay. In other embodiments, the assay is a cell-based assay.

As used herein, the term “modulate” encompasses “increase” and “decrease.” In some embodiments, of particular interest are agents which inhibit sulfatase activity, and/or which reduce a level of a subject sulfatase polypeptide in a cell, and/or which reduce a level of a subject sulfatase mRNA in a cell and/or which reduce release of a subject sulfatase from a eukaryotic cell. Such agents are of interest as candidates for treating cancers. In other embodiments, agents of interest are those that increase sulfatase activity; such agents are of interest as candidates for treating disorders amenable to treatment by increasing angiogenesis, e.g., ischemic conditions.

The terms “candidate agent,” “agent”, “substance” and “compound” are used interchangeably herein. Candidate agents encompass numerous chemical classes, typically synthetic, semi-synthetic, or naturally-occurring inorganic or organic molecules. Candidate agents may be small organic compounds having a molecular weight of more than 50 and less than about 2,500 daltons. Candidate agents may comprise functional groups necessary for structural interaction with proteins, particularly hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4° C. and 40° C. Incubation periods are selected for optimum activity, but may also be optimized to facilitate rapid high-throughput screening. Typically between 0.1 and 1 hour will be sufficient.

Methods for Identifying Agents that Modulate Sulfatase Activity

The present invention provides methods of identifying agents that modulate an enzymatic activity of a sulfatase polypeptide of the invention. The term “modulate” encompasses an increase or a decrease in the measured sulfatase activity when compared to a suitable control.

The method generally comprises:
a) contacting a test agent with a sample containing a sulfatase polypeptide; and
b) assaying a sulfatase activity of the sulfatase polypeptide in the presence of the substance. An increase or a decrease in sulfatase activity in comparison to sulfatase activity in a suitable control (e.g., a sample comprising a sulfatase polypeptide in the absence of the substance being tested) is an indication that the substance modulates an enzymatic activity of the sulfatase.

An “agent which modulates a sulfatase activity of a sulfatase polypeptide”, as used herein, describes any molecule, e.g. synthetic or natural organic or inorganic compound, protein or pharmaceutical, with the capability of altering a sulfatase activity of a sulfatase polypeptide, as described herein. Generally a plurality of assay mixtures is run in parallel with different agent concentrations to obtain a differential response to the various concentrations. Typically, one of these concentrations serves as a negative control, i.e. at zero concentration or below the level of detection. Sulfatase activity can be measured using any kinase assay known in the art.

Any sulfated compound that, upon cleavage of the sulfate group by the sulfatase activity, results in a change in absorption, fluorescence or other physical property amenable to detection, is suitable for use in a subject assay. Suitable substrates include, but are not limited to, 4-methylumbelliferyl sulfate; p-nitrophenyl sulfate; 4-methylumbelliferyl-α-D-N-acetylglucosamide-6-sulfate or 4-methylumbelliferyl-glucosamine-6-sulfate or conjugates containing these derivatives; any sulfated sugar or assembly of sugars related to heparan sulfate, including fragments of heparin or heparan sulfate; and any sulfated compound in which the sulfate is radiolabeled.

In certain embodiments, a substrate comprising a ³⁵S label is used. Release of ³⁵S is measured using any appropriate assay, e.g., scintillation counting, and the like.

In other embodiments, the substrate comprises a sulfated moiety that provides a detectable signal once the sulfate is released by action of the sulfatase. In a particular embodiment, the substrate is 4-methylumbelliferyl-sulfate. The reaction product of the action of a subject sulfatase on 4-methylumbelliferyl sulfate is 4-methylumbelliferone, which is a fluorescent compound. The product 4-methylumbelliferone is detected by an excitation wavelength of about 360 nm, whereupon the product emits at about 460 nm. Generally, the reaction includes 4-methylumbelliferyl-sulfate at about 10 mM, and 10 mM lead acetate. The reaction is carried out at 37° C. If desired, the reaction is stopped by addition of an excess of 0.5 M Na₂CO₃/NaHCO₃, pH 10.7. Sulfatase activity is detected by measuring fluorescence. This assay is particularly suited to a high through-put format.

An agent which modulates a sulfatase activity of a subject polypeptide increases or decreases the activity at least about 10%, at least about 15%, at least about 20%, at least about 25%, more preferably at least about 50%, more preferably at least about 100%, or 2-fold, more preferably at least about 5-fold, more preferably at least about 10-fold or more when compared to a suitable control.

Agents that increase or decrease a sulfatase activity of a subject polypeptide to the desired extent may be selected for further study, and assessed for cellular availability, cytotoxicity, biocompatibility, etc.

Of particular interest in some embodiments are agents that decrease a sulfatase activity of a subject polypeptide. Maximal inhibition of sulfatase activity is not always necessary, or even desired, in every instance to achieve a therapeutic effect. Agents which decrease a sulfatase activity of a subject polypeptide may find use in reducing angiogenesis stimulated by a tumor cell and thus may be useful in treating cancers.

Of particular interest in some embodiments are agents that increase a sulfatase activity of a subject polypeptide. Agents which increase a sulfatase activity of a subject polypeptide may find use in increasing angiogenesis and thus may be useful in treating ischemic conditions.

Cell-Based Methods

Cell-based methods include methods of detecting an agent that modulates a level of a subject sulfatase mRNA and/or subject sulfatase polypeptides, and methods for detecting an agent that modulates release of a subject sulfatase from a eukaryotic cell.

A candidate agent is assessed for any cytotoxic activity it may exhibit toward the cell used in the assay, using well-known assays, such as trypan blue dye exclusion, an MTT ([3-(4,5-dimethylthiazol-2-yl)-2,5-diphenyl-2H-tetrazolium bromide]) assay, and the like. Agents that do not exhibit cytotoxic activity are considered candidate agents.

The cells used in the assay are usually mammalian cells, including, but not limited to, rodent cells and human cells. The cells may be primary cell cultures or may be immortalized cell lines.

Methods of Detecting Agents that Modulate a Level of Sulfatase mRNA and/or Sulfatase Polypeptide

A wide variety of cell-based assays may be used for identifying agents which modulate levels of sulfatase mRNA and for identifying agents that modulate release of a sulfatase from a eukaryotic cell, using, for example, a mammalian cell transformed with a construct comprising a sulfatase-encoding cDNA such that the cDNA is overexpressed, or, alternatively, a construct comprising a sulfatase promoter operably linked to a reporter gene.

Accordingly, the present invention provides a method for identifying an agent, particularly a biologically active agent, that modulates a level of sulfatase expression in a cell, the method comprising: combining a candidate agent to be tested with a cell comprising a nucleic acid which encodes a sulfatase polypeptide; and determining the effect of said agent on sulfatase expression. “Modulation” of sulfatase expression levels includes increasing the level and decreasing the level of sulfatase mRNA and/or sulfatase polypeptide encoded by the sulfatase polynucleotide when compared to a control lacking the agent being tested. An increase or decrease of about 1.25-fold, usually at least about 1.5-fold, usually at least about 2-fold, usually at least about 5-fold, usually at least about 10-fold or more, in the level (i.e., an amount) of sulfatase mRNA and/or polypeptide following contacting the cell with a candidate agent being tested, compared to a control to which no agent is added, is an indication that the agent modulates sulfatase expression.

Sulfatase mRNA and/or polypeptide whose levels are being measured can be encoded by an endogenous sulfatase polynucleotide, or the sulfatase polynucleotide can be one that is comprised within a recombinant vector and introduced into the cell, i.e., the sulfatase mRNA and/or polypeptide can be encoded by an exogenous sulfatase polynucleotide. For example, a recombinant vector may comprise an isolated sulfatase transcriptional regulatory sequence, such as a promoter sequence, operably linked to a reporter gene (e.g,. β-galactosidase, CAT, luciferase, or other gene that can be easily assayed for expression). In these embodiments, the method for identifying an agent that modulates a level of sulfatase expression in a cell, comprises: combining a candidate agent to be tested with a cell comprising a nucleic acid which comprises a sulfatase gene transcriptional regulatory element operably linked to a reporter gene; and determining the effect of said agent on reporter gene expression. A recombinant vector may comprise an isolated sulfatase transcriptional regulatory sequence, such as a promoter sequence, operably linked to sequences coding for a sulfatase polypeptide; or the transcriptional control sequences can be operably linked to coding sequences for a sulfatase fusion protein comprising sulfatase polypeptide fused to a polypeptide which facilitates detection. In these embodiments, the method comprises combining a candidate agent to be tested with a cell comprising a nucleic acid which comprises a sulfatase gene transcriptional regulatory element operably linked to a sulfatase polypeptide-coding sequence; and determining the effect of said agent on sulfatase expression, which determination can be carried out by measuring an amount of sulfatase mRNA, sulfatase polypeptide, or sulfatase fusion polypeptide produced by the cell.

Cell-based assays generally comprise the steps of contacting the cell with an agent to be tested, forming a test sample, and, after a suitable time, assessing the effect of the agent on sulfatase expression. A control sample comprises the same cell without the candidate agent added. Sulfatase expression levels are measured in both the test sample and the control sample. A comparison is made between sulfatase expression level in the test sample and the control sample. Sulfatase expression can be assessed using conventional assays. For example, when a mammalian cell line is transformed with a construct that results in expression of sulfatase, sulfatase mRNA levels can be detected and measured, as described above, or sulfatase polypeptide levels can be detected and measured, as described above. A suitable period of time for contacting the agent with the cell can be determined empirically, and is generally a time sufficient to allow entry of the agent into the cell and to allow the agent to have a measurable effect on sulfatase mRNA and/or polypeptide levels. Generally, a suitable time is between 10 minutes and 24 hours, more typically about 1-8 hours.

Methods of measuring sulfatase mRNA levels are known in the art, several of which have been described above, and any of these methods can be used in the methods of the present invention to identify an agent which modulates sulfatase mRNA level in a cell, including, but not limited to, a PCR, such as a PCR employing detectably labeled oligonucleotide primers, and any of a variety of hybridization assays. Similarly, sulfatase polypeptide levels can be measured using any standard method, several of which have been described herein, including, but not limited to, an immunoassay such as ELISA, for example an ELISA employing a detectably labeled antibody specific for a sulfatase polypeptide.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used.

The screening methods may be designed a number of different ways, where a variety of assay configurations and protocols may be employed, as are known in the art. For example, one of the components may be bound to a solid support, and the remaining components contacted with the support bound component. The above components of the method may be combined at substantially the same time or at different times. Incubations are performed at any suitable temperature, typically between 4 and 40° C. Incubation periods are selected for optimum activity, but may also be optimized to facilitate rapid high-throughput screening. Typically between 0.1 and 1 hours will be sufficient. Following the contact and incubation steps, the subject methods will generally, though not necessarily, further include a washing step to remove unbound components, where such a washing step is generally employed when required to remove label that would give rise to a background signal during detection, such as radioactive or fluorescently labeled non-specifically bound components. Following the optional washing step, the presence of bound complexes will then be detected.

A variety of different candidate agents may be screened by the above methods. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Methods of Detecting Agents that Modulate Release of a Subject Sulfatase from a Eukaryotic Cell

Methods for identifying agents that modulate release of a sulfatase from a eukaryotic cell generally comprise contacting a cell that normally produces a subject sulfatase with a test agent, and determining the effect, if any, on release of the subject sulfatase.

“Modulation” of release of a subject sulfatase from a eukaryotic cell includes increasing the level and decreasing the level of release of a subject sulfatase from a eukaryotic cell when compared to a control lacking the agent being tested. An increase or decrease of about 1.25-fold, usually at least about 1.5-fold, usually at least about 2-fold, usually at least about 5-fold, usually at least about 10-fold or more, in the level (i.e., an amount) of sulfatase mRNA and/or polypeptide following contacting the cell with a candidate agent being tested, compared to a control to which no agent is added, is an indication that the agent modulates release of a subject sulfatase from a eukaryotic cell.

Cell-based assays generally comprise the steps of contacting the cell with an agent to be tested, forming a test sample, and, after a suitable time, assessing the effect of the agent on release of a subject sulfatase from a eukaryotic cell. A control sample comprises the same cell without the candidate agent added. Release of a subject sulfatase from a eukaryotic cell is measured in both the test sample and the control sample. A comparison is made between release of a subject sulfatase from a eukaryotic cell in the test sample and the control sample. Release of a subject sulfatase from a eukaryotic cell can be assessed using conventional assays to measure sulfatase activity. For example, when a mammalian cell line is transformed with a construct that results in expression of sulfatase, sulfatase enzymatic activity released from the cell can be detected and measured, as described above, or sulfatase polypeptide levels can be detected and measured, as described above. A suitable period of time for contacting the agent with the cell can be determined empirically, and is generally a time sufficient to allow entry of the agent into the cell (if necessary), or any other interaction wuth the cell, e.g., with cell-surface components) and to allow the agent to have a measurable effect on sulfatase release. Generally, a suitable time is between 10 minutes and 24 hours, more typically about 1-8 hours.

Agents

The invention further provides agents identified using a screening assay of the invention, and compositions comprising the agents, including pharmaceutical compositions. The subject compositions can be formulated using well-known reagents and methods. In some embodiments, compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Nucleic Acid and Polypeptide Therapeutic Compositions

The nucleic acid compositions and polypeptide compositions of the subject invention also find use as therapeutic agents in situations where one wishes to enhance sulfatase activity in a host, particularly the activity of the subject polypeptides, or to provide sulfatase activity at a particular anatomical site.

In some embodiments, a subject sulfatase is provided in a pharmaceutical composition with a pharmaceutically acceptable excipient. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

The subject genes, gene fragments, or the encoded proteins or protein fragments are useful in therapy to treat disorders associated with an activity of a subject sulfatase. Expression vectors may be used to introduce the gene into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

The gene or protein may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into skin cells.

In yet other embodiments of the invention, the active agent is an agent that modulates, and generally decreases or down regulates, the expression of the gene encoding the target protein in the host. For example, antisense molecules can be used to down-regulate expression of the subject genes in cells. The anti-sense reagent may be antisense oligonucleotides (ODN), particularly synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996), Nature Biotechnol. 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra, and Milligan et al., supra.) Preferred oligonucleotides are chemically modified from the native phosphodiester structure, in order to increase their intracellular stability and binding affinity. A number of such modifications have been described in the literature, which modifications alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The β-anomer of deoxyribose may be used, where the base is inverted with respect to the natural α-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 9523225, and Beigelman et al. (1995), Nucl. Acids Res. 23:4434-42). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al. (1995), Appl. Biochem. Biotechnol. 54:43-56.

Therapeutic Methods

The instant invention provides various therapeutic methods. In some embodiments, methods of regulating, including modulating and inhibiting, enzymatic activity of the subject proteins are provided. The subject methods find use in the treatment of a variety of different disease conditions, including, but not limited to, cancer; inflammation; disorders amenable to treatment by increasing angiogenesis, such as ischemic disorders; and thrombosis.

The host, or patient, may be from any mammalian species, e.g. primate sp., particularly humans; rodents, including mice, rats and hamsters; rabbits; equines, bovines, canines, felines; etc. Animal models are of interest for experimental investigations, providing a model for treatment of human disease.

As used herein, the term “agent” refers to a substance that modulates a level of enzymatically active subject sulfatase. In some embodiments, an agent is one identified by a screening assay of the invention. “Modulating a level of enzymatically active subject sulfatase” includes increasing or decreasing enzymatic activity of a subject sulfatase; increasing or decreasing a level of enzymatically active sulfatase protein; and increasing or decreasing a level of mRNA encoding enzymatically active subject sulfatase. In some embodiments, an agent is a subject sulfatase, where the subject sulfatase itself is administered to an individual. In some embodiments, an agent is an antibody specific for a subject sulfatase.

Methods of Reducing Tumor Growth

Disease conditions amenable to treatment by reducing an activity of a subject sulfatase and/or reducing a level of a subject sulfatase polypeptide or mRNA include those disease conditions associated with or resulting from the promotion of angiogenesis by a tumor. Thus, the subject methods are useful for reducing tumor-induced angiogenesis. In some embodiments, methods are provided for treating cancer. In some of these embodiments, methods are provided for reducing tumor growth. In other embodiments, methods are provided for reducing release of differentiation factors from the ECM.

Methods of reducing tumor growth, methods of reducing tumor-induced angiogenesis, and methods of reducing subject sulfatase activity, generally comprise administering to an individual an agent that reduces a level of enzymatically active subject sulfatase. An effective amount of an agent reduces the level of enzymatically active sulfatase by at least about 10%, at least about 20%, at least about 30%, at least about 40%, at least about 50%, or more, when compared to a suitable control. An effective amount of an agent reduces tumor growth by at least about 10%, at least about 20%, at least about 30%, at least about 40%, at least about 50%, or more, when compared to a suitable control.

Methods of reducing release of factors, such as growth factors and differentiation factors, from ECM are provided. The methods generally comprise administering to an individual an effective amount of an agent that reduces a level of enzymatically active subject sulfatase, where a reduction in the level of enzymatically active sulfatase results in a reduction of release of factor from the ECM adjacent to or surrounding the tumor.

Differentiation and growth factors include, but are not limited to, a fibroblast growth factor (FGF), a heparin-binding EGF-like growth factor, a hepatocyte growth factor, a member of the Wnt family of secreted glycoproteins, vascular endothelial growth factor (VEGF), platelet-derived growth factor (PDGF), a transforming growth factor (TGF), e.g., TGF-β, a bone morphogenetic protein, GM-CSF, and hepatocyte growth factor. In some embodiments, a factor released from the ECM by a subject sulfatase is a factor that binds heparan sulfate. In some embodiments, a factor-released from the ECM by a subject sulfatase is an angiogenic factor.

Tumors which may be treated using the methods of the instant invention include carcinomas, e.g. colon, prostate, breast, melanoma, ductal, endometrial, stomach, pancreactic, mesothelioma, dysplastic oral mucosa, invasive oral cancer, non-small cell lung carcinoma, transitional and squamous cell urinary carcinoma, etc.; neurological malignancies, e.g. neuroblastoma, glioblastoma, astrocytoma, gliomas, etc.; hematological malignancies, e.g. childhood acute leukaemia, non-Hodgkin's lymphomas, chronic lymphocytic leukaemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, etc.; and the like.

Whether tumor cell growth is inhibited or reduced can be assessed by any means known in the art, including, but not limited to, measuring tumor size; determining whether tumor cells are proliferating, e.g., by using a ³H-incorporation assay; and/or counting tumor cells.

Methods for Reducing Inflammation

In some embodiments, the invention provides methods of reducing inflammation, comprising increasing a level of enzymatically active subject sulfatase. Sulfatases act to remove a sulfate group from carbohydrate moieties of selectin ligands. Once a sulfate group is removed from the selectin ligand (e.g. from N-acetylglucosamine 6-sulfate), binding of the selectin to the ligand is reduced, and binding between an immune cell which a selectin on its surface to an selectin ligand on, e.g., the surface of an endothelial cell, is reduced. Accordingly, removal of a sulfate group from a selectin ligand reduces inflammation. In some embodiments, the methods comprise administering a subject sulfatase to an individual. In other embodiments, the methods comprise administering an agent (e.g., an agent identified by a screening method described above) to an individual, wherein said agent is one that increases a level of enzymatically active subject sulfatase in the individual. A therapeutically effective amount an agent is an amount sufficient to remove sulfate moieties from a substantial proportional number of ligands so that inflammation can either be prevented or ameliorated. Thus, “treating” as used herein in the context of inflammation shall mean preventing or ameliorating inflammation and/or symptoms associated with inflammation.

In determining the dose of sulfatases or agents to be administered, it must be kept in mind that one does not wish to completely remove all sulfates. In order for a normal healing process to proceed, at least some of the white blood cells or neutrophils must be brought into the tissue in the areas where the wound, infection or disease state is occurring. The amount of the sulfatases or agent administered is adjusted based on the particular needs of the patient while taking into consideration a variety of factors such as the type of disease that is being treated.

The subject sulfatases and/or agents are useful to treat a wide range of diseases, including diseases such as rheumatoid arthritis, asthma, adult respiratory distress syndrome, sarcoidosis, hypersensitivity pneumonitis multiple sclerosis, allograft rejection, and the spread of lymphomas to cutaneous sites. The compositions of the invention should be applicable to treat any disease state wherein the immune system turns against the body causing the white cells to accumulate in the tissues to the extent that they cause tissue damage, swelling, inflammation and/or pain. The inflammation of rheumatoid arthritis, for example, is created when large numbers of white blood cells quickly enter the joints in the area of disease and attack the surrounding tissues.

Formulations of sulfatases and/or agent are administered to prevent the undesirable aftereffects of tissue damage resulting from heart attacks. When a heart attack occurs and the patient has been revived, such as by the application of anticoagulants or thrombolytic (e.g., tPA), the endothelial lining where a clot was formed has often suffered damage. When the antithrombotic has removed the clot, the damaged tissue beneath the clot and other damaged tissue in the endothelial lining which has been deprived of oxygen become activated. The white blood cells possess L-selectin. The receptors adhere to ligand molecules on the surface of activated endothelial cells. The ligand molecules may be induced to the surface of the endothelial cells by activation. Large numbers of white blood cells are quickly captured and brought into the tissue surrounding the affected area, resulting in inflammation, swelling and necrosis which thereby decreases the likelihood of survival of the patient.

In addition to treating patients suffering from the trauma resulting from heart attack, patients suffering from actual physical trauma could be treated with formulations of the invention in order to relieve the amount of inflammation and swelling which normally result after an area of the body is subjected to severe trauma. This is most preferably done by local injection of sulfatases and/or agent to the area subjected to trauma. Also, patients suffering from hemorrhagic shock could be treated to alleviate inflammation associated with restoring blood flow. Other disease states which might be treatable using formulations of the invention include various types of arthritis, various chronic inflammatory conditions of the skin, insulin-dependent diabetes, and adult respiratory distress syndrome. After reading the present disclosure, those skilled in the art will recognize other disease states and/or symptoms which might be treated and/or mitigated by the administration of formulations of the present invention.

Methods of Increasing Angiogenesis

In some embodiments, the invention provides methods for increasing angiogenesis. The methods generally involve administering to a mammal having a condition amenable to treatment by increasing angiogenesis an effective amount of a subject sulfatase. In many embodiments, the subject sulfatase will be administered locally to an anatomical site.

Examples of conditions and diseases amenable to treatment according to the method of the invention include any condition associated with an obstruction of a blood vessel, e.g., obstruction of an artery, vein, or of a capillary system. Specific examples of such conditions or disease include, but are not necessarily limited to, coronary occlusive disease, carotid occlusive disease, arterial occlusive disease, peripheral arterial disease, atherosclerosis, myointimal hyperplasia (e.g., due to vascular surgery or balloon angioplasty or vascular stenting), thromboangiitis obliterans, thrombotic disorders, vasculitis, and the like. Examples of conditions or diseases that can be prevented using the methods of the invention include, but are not necessarily limited to, any of a variety of ischemic conditions (e.g., myocardial ischemia, limb ischemia, ischemia associated with stroke), heart attack (myocardial infarction) or other vascular death, stroke, death or loss of limbs associated with decreased blood flow, and the like.

Thus, the invention provides methods of treating an ischemic condition. Administration of an effective amount of a subject sulfatase results in an increase in angiogenesis, and as a result, an increased blood supply to an ischemic tissue. Following administration of a subject sulfatase, blood supply (blood flow) to the ischemic tissue is increased by at least about 10%, at least about 20%, at least about 30%, at least about 50%, at least about 75%, or at least about 100%, or more when compared to a suitable control. Whether the blood supply to an ischemic tissue is increased can be measured by any method known in the art, including, but not limited to, thermography; infrared recorder; transcutaneous PO₂, transcutaneous PCO₂, laser Doppler, Doppler waveform, ankle brachial index, pulse volume recording, toe pressure, duplex waveform, magnetic resonance imaging profile, isotope washout, and NAD/NADH fluorometry. Such methods are well known in the art and have been described in numerous publications, including, e.g., Lazarus et al. ((1994) Arch. Dermatol. 130:491) and references cited therein.

Whether angiogenesis is increased can be determined using any known assay. Whether angiogenesis is increased can be determined using any method known in the art, including, e.g., stimulation of neovascularization into implants impregnated with relaxin; stimulation of blood vessel growth in the cornea or anterior eye chamber; stimulation of endothelial cell proliferation, migration or tube formation in vitro; and the chick chorioallantoic membrane assay; the hamster cheek pouch assay; the polyvinyl alcohol sponge disk assay. Such assays are well known in the art and have been described in numerous publications, including, e.g., Auerbach et al. ((1991) Pharmac. Ther. 51: 1-11), and references cited therein.

Methods of Reducing Thrombosis

The invention further provides methods of reducing thrombosis in an individual, the methods generally involving administering an effective amount of an inhibitor of a subject sulfatase. In some embodiments, the inhibitor is a small molecule inhibitor of sulfatase activity of a subject sulfatase. In other embodiments, the inhibitor is an antibody specific for a subject sulfatase, which antibody inhibits the sulfatase activity, either directly or by effecting removal of the sulfatase.

Formulations, Dosages, and Routes of Administration

As mentioned above, an effective amount of the active agent (e.g., small molecule, anti-sulfatase antibody, or a subject sulfatase) is administered to the host, where “effective amount” means a dosage sufficient to produce a desired result. In some embodiments, the desired result is at least a reduction in enzymatic activity of a subject sulfatase as compared to a control. In other embodiments, the desired result is an increase in the level of enzymatically active sulfatase (in the individual, or in a localized anatomical site in the individual), as compared to a control.

Typically, the compositions of the instant invention will contain from less than 1% to about 95% of the active ingredient, preferably about 10% to about 50%. Generally, between about 100 mg and 500 mg will be administered to a child and between about 500 mg and 5 grams will be administered to an adult. Administration is generally by injection and often by injection to a localized area. The frequency of administration will be determined by the care given based on patient responsiveness. Other effective dosages can be readily determined by one of ordinary skill in the art through routine trials establishing dose response curves.

In order to calculate the amount of sulfatase enzyme, those skilled in the art could use readily available information with respect to the amount of enzyme necessary to remove a given amount of sulfatase. For example, if a given enzyme has an activity such that one unit of the enzyme removes 1 micromole/min. of SO₄ from a substrate at physiological pH, then one would administer from 1 to 10 units intravenously to a 70 kg. human for therapeutic purposes. The amount of an agent necessary to increase a level of enzymatically active subject sulfatase can be calculated from in vitro experimentation. For example, by calculating the amount of agent necessary to increase removal of sulfate groups from a given amount of substrate and estimating the amount of such substrate (or its in vivo equivalent) within the area to be treated, an amount of agent to be administered can be determined. The amount of agent will, of course, vary depending upon the particular agent used.

In the subject methods, the active agent(s) may be administered to the host using any convenient means capable of resulting in the desired inhibition of sulfatase activity. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of the agents can be achieved in various ways, including oral, buccal, rectal, parenteral, intraperitoneal, intradermal, transdermal, intracheal, etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the chlorate/selenate and/or sulfatase adequate to achieve the desired state in the subject being treated.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the agent is a polypeptide, polynucleotide, analog or mimetic thereof, e.g. antisense composition, it may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the therapeutic DNA, then bombarded into skin cells.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as inflammation and pain associated therewith. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

The various sulfatases and agent of the present invention can be used by themselves, with each other, or in combination with pharmaceutically acceptable excipient materials as described above.

Kits with unit doses of the active agent, usually in oral or injectable doses, are provided. In such kits, in addition to the containers containing the unit doses will be an informational package insert describing the use and attendant benefits of the drugs in treating pathological condition of interest. Preferred compounds and unit doses are those described herein above.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Identification of Novel Human Sulfatase-Encoding Nucleic Acid Molecules

HuSULF-1 and huSULF-2 sequences were derived based on a partial protein sequence (15 amino acids), and using a BLAST (i.e., tblastn) search of the NCBI public database to find expressed sequence tags that overlapped with the protein sequence. The new ESTs were then used to find additional corresponding ESTs and genomic sequences from public databases. A contig was assembled to yield a full-length cDNA. We were also able to identify from human ESTs and genomic sequences a full-length cDNA sequence corresponding to human sulf2, which is highly related to human sulf-1. From the cDNAs for the two genes, we derived predicted protein sequences. The nucleotide sequence of huSULF-1 cDNA is provided in FIGS. 1Ai and 1Aii; the amino acid sequence of huSULF-1 is provided in FIG. 1B. The nucleotide sequence of huSULF-2 cDNA is provided in FIGS. 2Ai and 2Aii; the amino acid sequence of huSULF-2 is provided in FIG. 2B.

Using a similar approach, we derived full-length sequences of mouse SULF-1 and mouse SULF-2. The nucleotide sequence of mouse SULF-1 cDNA is provided in FIGS. 3Ai and 3Aii; the amino acid sequence of mouse SULF-1 is provided in FIG. 3B. The nucleotide sequence of mouse SULF-2 cDNA is provided in FIGS. 4Ai and 4Aii; the amino acid sequence of mouse SULF-2 is provided in FIG. 4B.

### Example 2

### Determining the Frequency of Expression of huSULF-1 and huSULF-2 in Normal and Cancerous Tissues

Expressed Sequence Tags (EST)

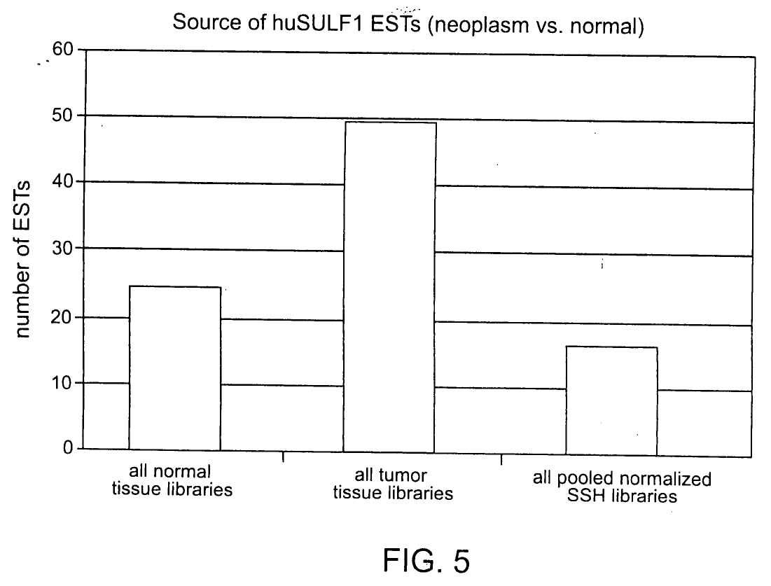

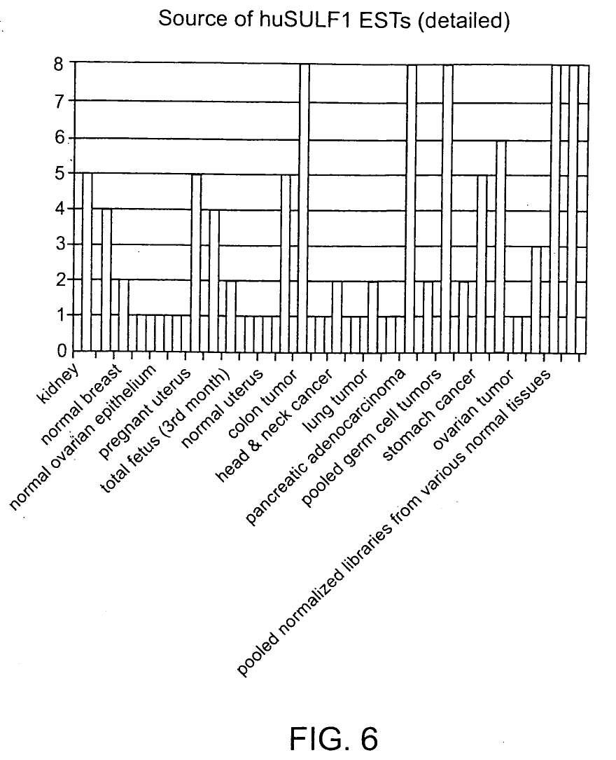

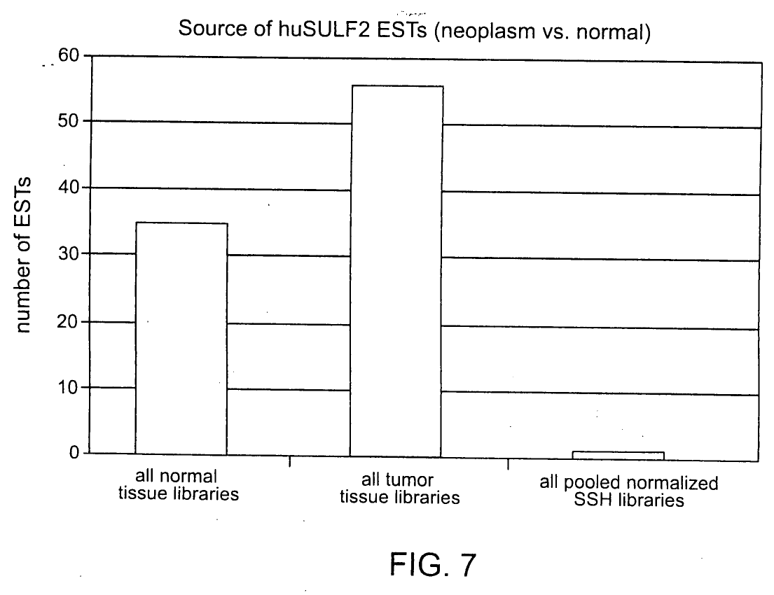

The electronic northerns were accomplished as follows. The Genbank huEST database was subjected to a BLAST search (blastn) with the full length cDNAs of human sulf-1 and human sulf-2 respectively. Only those hits with p<IE-100 (perfect matches) were collected (total of 98 for either huSULF). At this stringency there were no redundant ESTs that mapped to both isozymes. The source of each EST was determined by examining every single pertinent GenBank record and tabulating the results. Similar sources such as glioblastoma and brain cancer were pooled. The results are shown in FIGS. 5, 6, and 7. The results indicate that huSULF1 and huSULF2 are expressed at elevated levels in cancerous tissue, when compared to normal, non-cancerous tissue.

SAGE

Serial analysis of gene expression, or SAGE, is a technique designed to take advantage of high-throughput sequencing technology to obtain a quantitative profile of cellular gene expression. Essentially, the SAGE technique measures not the expression level of a gene, but quantifies a “tag” which represents the transcription product of a gene. A tag, for the purposes of SAGE, is a nucleotide sequence of a defined length, directly 3′-adjacent to the 3′-most restriction site for a particular restriction enzyme. As originally described, the length of the tag was nine bases, and the restriction enzyme NlaIII. Current SAGE protocols produce a ten to eleven base tag, and, although NlaIII remains the most widely used restriction enzyme, enzyme substitutions are possible. The data product of the SAGE technique is a list of tags, with their corresponding count values, and thus is a digital representation of cellular gene expression. Velculescu V E, Zhang L, Vogelstein B, Kinzler K W. Serial analysis of gene expression. Science. 1995 Oct. 20; 270(5235):484-7; and Zhang L, Zhou W, Velculescu V E, Kern S E, Hruban R H, Hamilton S R, Vogelstein B, Kinzler K W. Gene expression profiles in normal and cancer cells. Science. 1997 May 23; 276(5316):1268-72. There are currently approximately 3×10⁶ SAGE tags from about 80 libraries.

SAGE libraries were examined for the presence of huSULF2 sequences. Libraries corresponding to normal and cancerous tissues (both cell lines and tissue samples) were analyzed. The results are shown in Table 1. The number of total available SAGE tags is provided, as well as the number of available tags that contain huSULF2 sequence.

TABLE 1

| | Normal | Cancerous |
|---|---|---|
| BREAST | | |
| Total available | 136,256 | 279,790 |
| huSULF2 | 14 | 180 |
| COLON | | |
| Total available | 235,923 | 621,404 |
| huSULF2 | 15 | 196 |

The data provided in Table 1 indicate that both huSULF1 and huSULF2 are highly expressed in cancerous cells.

### Example 3

### SAGE Analysis of huSULF-1 and huSULF-2

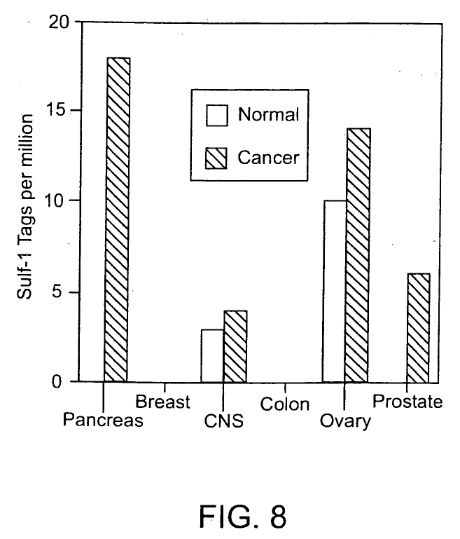

When SAGE analysis was applied to the human sulf-1 and sulf-2, there were striking findings. In the case of hsulf-1, significantly more tags were found in cancer tissue (normalized to specific tags per million of total tags) compared to normal tissue for both pancreas and prostate. The results are shown in FIG. 8.

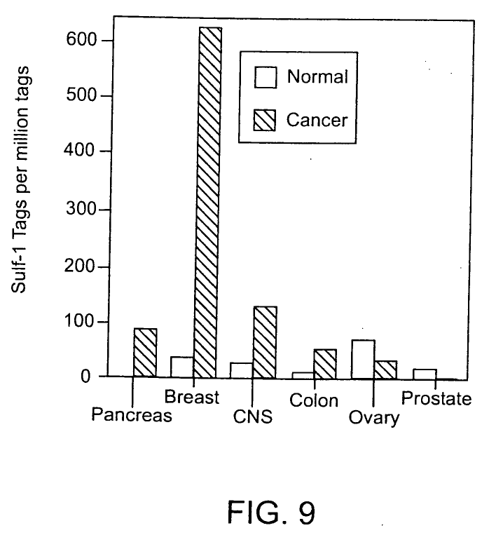

In the case of sulf-2, the findings were even more dramatic. For 4 different cancers (pancreas, breast, central nervous system, and colon), the normalized tag representation (based on specific tags per million of total tags) was significantly higher in the cancer tissue as compared to the normal counterpart tissue. The results were most dramatic for breast cancer. Here the expression in the cancer tissue was extremely high, about 6-fold higher than in any of the other cancer tissues, and furthermore the level in breast cancer tissue was 17-fold higher than in normal breast tissue. The results are shown in FIG. 9.

These results indicate the upregulation of sulf gene expression in human cancers, with one or the other sulf gene more important depending on the nature of the cancer. Thus, the sulf gene products—extracellular sulfatase enzymes—are appropriate targets for cancer therapy. Inhibition of these enzymes blocks the growth of tumors by preventing the release of growth factors or blocks the formation of new blood vessels associated with tumor growth (angiogenesis) and therefore prevents the growth and metastasis of the tumors.

### Example 4

### cDNA Cloning

Human SULF2

A 4286 bp cDNA was identified, and isolated from a human lung cDNA library and sequenced along both strands. This cDNA contains a 2613 bp open reading frame (ORF) that encodes an 870 amino acid polypeptide termed human SULF2. The human SULF2 gene is situated on human chromosome 20q12-13.2 since a genomic clone containing exons 11 through 20 of this gene has been localized to this region previously (Genbank accession no. AL034418). The nucleotide sequence of huSULF-2 cDNA is provided in FIGS. 10Ai and 10Aii; the amino acid sequence of huSULF-2 is provided in FIG. 10B.

Mouse SULF2

A cDNA encoding the mouse homologue of human SULF2 was identified in IMAGE clone 3155559 (Genbank accession no. AW763993) derived from a mouse mammary tumor. This clone was retrieved and DNA was prepped and sequenced along both strands. It was found to contain a 3613 bp cDNA containing a 2628 bp ORF encoding an 875 amino acid protein termed mouse SULF2 that is 94.6% identical to human SULF2 on the amino acid level (GCG-BESTFIT). The nucleotide sequence of mouse SULF-2 cDNA is provided in FIGS. 11Ai and 11Aii; the amino acid sequence of mouse SULF-2 is provided in FIG. 11B.

### Example 5

### Genomic Organization of the Human SULF2 Gene

Fragments of the human SULF2 cDNA were used to screen the Genbank nr and htgs databases for matching genomic fragments. The retrieved matches were then assembled using the Sequencher contig alignment software. Thus four contigs (I, II, III, and IV) were assembled that contain the entire huSULF2 cDNA as 21 exons. The concanated sequence is provided in SEQ ID NO:22. The three gaps separating the four contigs are indicated by trains of N (NNNNNNNNNNN). The length of these three gaps is presently unknown. The genomic organization of the gene was determined. The lengths, relative positions, and separating gaps of all 21 exons are shown in FIG. 12. Contig I is expected to contain regulatory elements (promotor and enhancer sequences) upstream of exon 1.

### Example 6

### Analysis of Protein Structure

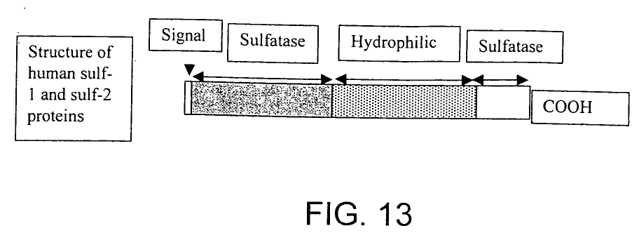

FIG. 13 shows the structure of huSULF-1 and huSULF-2 proteins. Human sulf-1 is 871 amino acids and human sulf-2 is 870 amino acids in length. Hu-SULF1 and huSULF-2 are 65% identical at the amino acid level. Both have cleavable signal sequences at the amino termini of the proteins: 1-22 amino acids for sulf-1 and 1-24 amino acids for sulf-2. This feature indicates that these enzymes are secreted from the cells of origin (in contrast to the lysosomal glucosamine-6-sulfatase enzyme) and are present in the extracellular space where they can act on extracellular heparan sulfate proteoglycans and related glycoconjugates. Following the signal sequences are “sulfatase” domains which extend to about amino acid 400. This “sulfatase” designation is based on a block analysis of the protein. In this region, the closest homologue is the lysosomal glucosamine-6-sulfatase, which shows about 49% identity at the amino acid level to sulf proteins over this region (24-400 amino acids). Thus the sulf proteins are glucosamine-6-sulfatase enzymes with activity against heparan sulfate glycosaminoglycans and related glycoconjugates.

Within the first sulfatase domains are cleavage sites for the furan/PACE protease processing enzymes. This cleavage occurs between residues 408 (arginine) and 409 (aspartic acid) and/or between 576 (arginine) and 577 (histidine) of hsulf-1. The cleavage occurs between 409 (arginine) and 410 (aspartic acid) and/or between 423 (arginine) and 424 (aspartic acid) and/or between 538 (arginine) and 539 (serine) and/or between 565 (arginine) and 566 (histidine) of hsulf-2. Cleavage is necessary for activity of the enzyme.

Following the first “sulfatase domain” are hydrophilic domains containing a high concentration of charged amino acids which are predominantly basic in nature. These domains are comprised of about 370 amino acids. The last domain (the second “sulfatase” domain) which extends to the carboxy terminus of the proteins (70 amino acids in length) is also homologous to the C-terminus of the lysosomal glucosamine-6-sulfatase enzyme. There is also significant homology with an O-GlcNAc transferase (Arabidopsis) in the second sulfatase domain. Thus, the first sulfatase domain is involved in cleavage of the sulfate moiety from glucosamine-6-sulfate structures within heparan sulfate glycosaminoglycans and other related glycoconjugates, whereas the second sulfatase domain is involved in substrate recognition of glucosamine and N-acetylglucosamine sugars.

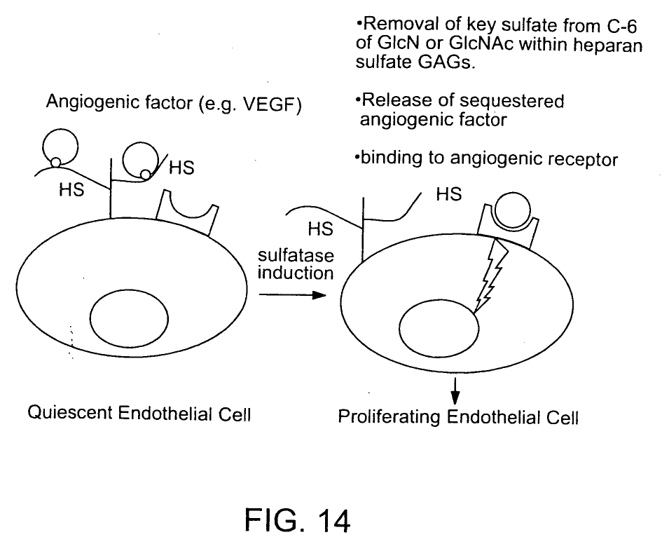

FIG. 14 presents a model of activity of a subject sulfatase. Subject sulfatases are extracellular enzymes that remove sulfate from the C-6 position of glucosamine (GlcN) or N-Acetyl glucosamine (GlcNAc) within heparan sulfate proteoglycans on the cell surface. The sulfatase releases growth factors/differentiation factors/angiogenic factors. An example of such a factor is vascular endothelial growth factor (VEGF). Release of VEGF makes it available to endothelial cells (EC), converting a quiescent (e.g., non-angiogenic) EC to a proliferating (e.g., angiogenic) EC.

### Example 7

### Expression of Hsulf-1 and Hsulf-2 in CHO Cells

Methods

Human sulf-1 (hsulf-1), hsulf-2 cDNA, mouse sulf-1 (msulf-1), and msulf-2 cDNAs were digested with XhoI and BamHI, HindIII and XhoI, NheI and HindIII, or HindIII and XhoI restriction enzymes, respectively and subcloned into the corresponding sites of pcDNA3.1/Myc-His(−) (Invitrogen Inc.). This 5.5 kb vector is designed for overproduction of recombinant proteins with a C-terminal tags consisting of a polyhistidine metal-binding tag and the myc epitope. Chinese hamster ovary cells (CHO) were grown in 10 cm dishes and transfected with 5 μg of pcDNA3.1/Myc-His(−)-hsulf-1, -hsulf-2, -msulf-1, or -msulf-2 using Lipofectamine and Plus reagent (Invitrogen Inc.) according to the manufacturer's instructions. DNA was mixed with Plus reagent and incubated for 15 minutes at room temperature.

The complexed DNA was combined with Lipofectamine reagent (diluted in OptiMEM (GIBCO BRL)) and incubated for 15 minutes at room temperature. The complexes were added to cells in culture dishes, and incubated at 37° C. at 5% CO₂ for 5 hours. After incubation, medium was replaced with OptiMEM. Cells were allowed to grow for an additional 48 hours, and the conditioned medium was collected. The samples were concentrated on a Centricon30 microconcentrator (Amicon), separated by electrophoresis on reducing SDS-8% polyacrylamide gels (ISC BioExpress), blotted to ProBlott™ (Applied Biosystems). The membranes were blocked for 1 hour with 5% non-fat milk and then incubated overnight with a 0.22 μg/ml dilution of anti-Myc antibody (Invitrogen) in 5% non-fat milk. Membranes were washed and incubated with horseradish peroxidase goat anti mouse IgG1 (0.4 μg/ml dilution) (Caltag) for 1 hour before enhanced chemiluminescence (ECL) detection reagents (Amersham Pharmacia).

Results

The 4 sulfatase fusion proteins were detected as a series of bands as follows (hsulf-1: 126, 61, 53 kDa) (hsulf-2: 126, 61 kDa), (msulf-1: 126, 61, 49, 40 kDa) and (msulf-2: 126, 71, 66 kDa).

### Example 8

### Verification of the Sulfatatase Activities of the Sulf Proteins

Methods

The 100-fold concentrated conditioned medium derived from each transfection of CHO cells was dialyzed into 50 mM HEPES, pH 8.0. The his-tagged fusion proteins were bound to a Ni-NTA resin (QIAGEN) by rotation at 4° C. over night, then washed with 50 mM HEPES (pH 8.0), 3 times. These resins were mixed with 10 mM 4-methylumbelliferyl-sulfate (a substrate for sulfatases), and 10 mM lead acetate, and total volume is 100 μl. The reaction mixtures were incubated at 37° C. for varying periods of time with termination of the reaction by addition of 100 μl of 0.5 M Na₂CO₃/NaHCO₃, pH10.7 to 20 μl of the reaction mixture. The fluorescence of 4-methylumbelliferone was measured on a Multi-Well Plate Reader CytoFluorII (PerSeptive Biosystems). The fluorescence was determined at an excitation wavelength of 360 nm and emission wavelength of 460 nm.

Result

Time-dependent sulfatase activity was detected for both the hsulf-1 and h-sulf-2 fusion proteins. The activity varied with the concentration of enzyme added, as demonstrated for hsulf-1. These results demonstrated unequivocally that the subject proteins possess sulfatase activity.

### Example 9

### Expression of Sulf Genes in Human Breast Cancer Tissues

Methods

The Rapid-Scan Gene Expression Panel (Origene Inc.) is a set of cDNAs prepared from 12 independent normal breast tissues (human) and 12 independent breast cancer patients. A 314-bp hsulf-2 cDNA product was amplified using the following PCR primers: sense 5′-GAAAAGAGGCAGATTCACGTCGTTTCCAG-3′ (SEQ ID NO:25), antisense 5′-ATCTGGTGCTTCTTTTGGGATGCGGGAG-3′ (SEQ ID NO:26). The conditions for denaturation, annealing, and extension of the template cDNA were respectively: 94° C. for 30 seconds, 55° C. for 30 seconds, 72° C. for 1 minute for 40 cycles. For each source of cDNA, PCR was performed at 4 different cDNA concentrations (1×, 10×, 100× and 1000×) using TITANIUM™ Taq DNA Polymerase (Clontech). The PCR products were then electrophoresed on 2% agarose gels, and visualized with ethidium bromide.

Results

Nine of 12 of the breast cancer specimens were positive for hsulf-2 expression whereas none (0 of 12) of the normal breast tissue samples were positive at any cDNA concentration. The results are shown in Table 2, below. The level of expression of estrogen receptor (ER) and progesterone receptor (PR) on breast cancer tissues is also shown.

TABLE 2

| | | | character- | Expression |
|---|---|---|---|---|
| lane | Tissue | Grade | istics | of hsulf-2 |
| 1 | Normal breast | | | − |
| 2 | Normal breast | | | − |
| 3 | Normal breast | | | − |
| 4 | Normal breast | | | − |
| 5 | Normal breast | | | − |
| 6 | Normal breast | | | − |
| 7 | Normal breast | | | − |
| 8 | Normal breast | | | − |
| 9 | Normal breast | | | − |
| 10 | Normal breast | | | − |
| 11 | Normal breast | | | − |
| 12 | Normal breast | | | − |
| 13 | Invasive mixed tubular | 5 | ER+ | − |
| | carcinoma | | PR+++ | |
| 14 | Invasive ductal carcinoma | 9 | ER+ | − |
| | | | PR+++ | |
| 15 | Invasive lobular carcinoma | 6 | ER++++++ | + |
| | | | PR+++++ | |
| 16 | Invasive ductal carcinoma | 7 | ER++ | − |
| | | | PR− | |
| 17 | Invasive ductal carcinoma | ? | ER++ | + |
| | | | PR− | |
| 18 | Invasive ductal carcinoma | 6 | ER+++ | + |
| | | | PR+ | |
| 19 | Invasive ductal carcinoma | 5 | ER++ | + |
| | | | PR+ | |
| 20 | Invasive ductal carcinoma | 6 | ER+ | + |
| | | | PR− | |
| 21 | Adenoid cystic carcinoma | — | ER++ | + |
| | | | PR+ | |
| 22 | Invasive ductal carcinoma | 5 | ER− | + |
| | | | PR− | |
| 23 | Ductal carcinoma in-situ | — | ER+ | + |
| | | | PR+/− | |
| 24 | Invasive ductal carcinoma | 8 | ER+ | + |
| | | | PR+ | |

It is evident from the data presented above that the instant invention provides sulfatases that are glucosamine-6-sulfatase enzymes with activity against heparan sulfate glycosaminoglycans and related glycoconjugates. The instant sulfatases are secreted from eukaryotic cells, and are expressed at higher than normal levels in cancerous tissue, compared to normal tissue. The instant invention also provides methods of assaying for sulfatase activity, which assay is readily adapted to a high throughput format.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. An isolated polypeptide having glucosamine-6-sulfatase activity.

2. The polypeptide according to claim 1, wherein said sulfatase is a human sulfatase.

3. The polypeptide according to claim 1, wherein said sulfatase comprises an amino acid sequence set forth in any one of SEQ ID NO: 03, 06, 09, 12, 15, and 18.

4. An isolated polynucleotide comprising a nucleotide sequence encoding a sulfatase having glucosamine-6-sulfatase activity.

5. The polynucleotide according to claim 4, wherein said polynucleotide comprises a nucleotide sequence that encodes a polypeptide comprising an amino acid sequence set forth in any one of SEQ ID NO: 03, 06, 09, 12, 15, and 18.

6. An expression cassette comprising a transcriptional initiation region functional in an expression host, a nucleic acid having a nucleotide sequence according to claim 4 under the transcriptional regulation of said transcriptional initiation region, and a transcriptional termination region functional in said expression host.

7. A cell comprising an expression cassette according to claim 6 as part of an extrachromosomal element or integrated into the genome of a host cell as a result of introduction of said expression cassette into said host cell, or the cellular progeny thereof.

8. A method of producing a sulfatase, said method comprising:
growing a cell according to claim 7, whereby said sulfatase is produced by said cell; and
isolating said sulfatase substantially free of other proteins.

9. A monoclonal antibody binding specifically to a sulfatase.

10. A method of identifying an agent that inhibits an enzymatic activity of a sulfatase, the method comprising:
a) contacting a sulfatase according to claim 1 with a test agent and a substrate for the sulfatase; and
b) determining the effect, if any, on glucosamine-6-sulfatase activity of the sulfatase.

11. The method according to claim 1Q, wherein the substrate is 4-methylumbelliferyl sulfate, and said determining is by measuring the amount of 4-methylumbelliferone reaction product produced.

12. A method of reducing tumor-induced angiogenesis in an individual having a tumor, the method comprising:
administering to the individual an effective amount of an agent that inhibits enzymatic activity of a sulfatase that releases an angiogenic factor from its association with extracellular matrix, thereby reducing angiogenesis.

13. The method according to claim 12, wherein said reduction in angiogenesis results in a reduction in tumor growth.

14. The method according to claim 12, wherein said administering is at or near the site of the tumor.

15. A method of treating an ischemic condition in an individual, the method comprising administering an effective amount of a sulfatase polypeptide according to claim 1 to the individual, wherein the sulfatase polypeptide increases angiogenesis, thereby treating the ischemic condition.

16. A method of detecting the presence of a cancerous cell in a tissue, the method comprising:
determining the level of an mRNA in a host tissue sample comprising a sequence that encodes a sulfatase according to claim 1; and
comparing the level of said mRNA in said host tissue sample to a control value, wherein an elevated level of sulfatase mRNA in a cell in the tissue sample, compared to a control, indicates the presence of a cancerous cell in the tissue.

17. The method of claim 16, wherein said determining is by amplifying a cDNA copy of the mRNA using specific primer oligonucleotides and a DNA polymerase.

18. A method of reducing tumor growth in an individual having a tumor, the method comprising:
administering to the individual an effective amount of an agent that inhibits enzymatic activity of a sulfatase according to claim 1, wherein said sulfatase releases a growth factor from extracellular matrix, and wherein inhibition of enzymatic activity of said sulfatase reduces tumor growth.

19. The method of claim 18, wherein said administering is at or near the site of the tumor.

20. A method of detecting the presence of a cancerous cell in an individual, the method comprising:
determining the level of a sulfatase polypeptide according to claim 1 in a biological sample from said individual; and
comparing the level of said polypeptide in said sample to a control value, wherein an elevated level of said sulfatase in the biological sample, compared to a control, indicates the presence of a cancerous cell in said individual.

21. The method according to claim 20, wherein said determining is by contacting said sample with an antibody specific for said sulfatase polypeptide.

22. The method according to claim 20, wherein said determining is by contacting said sample with a substrate for said sulfatase, such that a detectable product is produced in the presence of said sulfatase.
